(19) United States
(12) Patent Application Publication
(10) Pub. No.: US 2017/0056582 A1
(43) Pub. Date: Mar. 2, 2017

(54) METERING DEVICE FOR DISPENSING PHARMACEUTICAL FLUID FROM A RESERVOIR HAVING A SPINDLE ROD FOR DISPLACEMENT OF THE PISTON

(71) Applicant: MeaMedical AG, Riken (CH)

(72) Inventor: Hanspeter NIKLAUS, Riken (CH)

(73) Assignee: MeaMedical AG, Riken (CH)

(21) Appl. No.: 15/350,260

(22) Filed: Nov. 14, 2016

Related U.S. Application Data
(63) Continuation of application No. PCT/EP2015/058194, filed on Apr. 15, 2015.
Application No. PCT/EP2015/058194 is a continuation-in-part of application No. PCT/EP2014/074907, filed on Nov. 18, 2014.
Application No. PCT/EP2014/074907 is a continuation-in-part of application No. PCT/EP2014/059889, filed on May 14, 2014.

(51) Int. Cl. A61M 5/145; A61M 5/142

(52) U.S. Cl. CPC A61M 5/14566; A61M 5/14248; A61M 2005/14252; A61M 2005/14268; A61M 2005/14573; A61M 2005/16863

(57) ABSTRACT

A metering device for delivering a pharmaceutical fluid, includes a reusable part and a consumable part. The reusable part includes a housing, a control unit, a driving device, and a coupling member driven by an output wheel for transmitting a drive output to a spindle device. The consumable part includes the fluid reservoir and a piston which can be advanced by the spindle device, with the spindle device disposed on the consumable part and having a spindle, and the spindle device coupled by the coupling member to the reusable part. A force sensor disposed on the reusable part is provided for indirect pressure measurement. The metering device is configured such that after partial or complete filling, the spindle device is in the retracted state. The coupling member is axially slidably mounted and the output wheel is supported by the force sensor on a fixed base.

### TECHNICAL FIELD

The invention relates to a metering device for dispensing a liquid medication into the body of a user.

### BACKGROUND

Portable injection and/or infusion devices are used for administering liquid medications, in particular insulin for treatment of diabetes. The liquid medication is delivered continuously or semi-continuously by means of a metering device, comprising a driving device for a plunger and a reservoir containing the fluid. The plunger is advanced into the reservoir, displacing the liquid medication in the reservoir and administering it to the user. Such devices are used as pump devices and as manually operated pens in the treatment of insulin. It is true of both injection pens and insulin pumps that these devices must be as compact as possible, while being reliable and easy to operate and safe for the user.

The D-TRONplus insulin pump from Roche Diabetes Care GmbH is one example of such a metering device. It has a spindle device, which is mounted permanently on the pump and is formed from two telescoping displacement stages and a rotating driving stage that is axially secured. The first displacement stage, which is movable against the plunger of the reservoir, can only execute a forward movement. In movement of the first displacement stage, it abuts against the second displacement stage, wherein the second displacement stage is entrained rotatingly by the driving stage. After advancing the first displacement stage, the movement of the second displacement stage takes place. The second displacement stage executes only a translational movement, and in doing so, encounters the driving stage, which is still being driven to rotate. The spindle device of the D-TRONplus insulin pump is described in DE 19717107 B4. In addition, a method and a device for monitoring the pressure in the reservoir of the D-TRONplus insulin pump are also described in DE 19840992 A1. This document describes a drive for a plunger, which is in a floating mount in a fixed pump housing by means of O-rings and in a direction opposite the forward direction of the plunger, wherein the drive comprises its own housing as a displacement platform, a motor, a gear and the telescoping spindle device. At least the spindle device is disposed in the displacement platform; preferably both the motor gear as the driving device and also the spindle device are integrated into the displacement platform.

DE 19840992 A1 proposes that the drive in a floating mount should be supported on the housing via a force sensor. An axial reactive force can be measured indirectly on the spindle device via the force sensor, wherein the reaction force is proportional to the pressure in the reservoir. FIG. 1 shows that the displacement platform on the pump housing is sealed by means of a pair of O-rings. The elastic O-rings also serve to provide a floating mount of the displacement platform in the fixed pump housing. The sealing sites have relatively unfavorable large diameters. These exterior sealing points disposed between the displacement platform and the pump housing are not suitable for an accurate determination of pressure in the reservoir. When the fluid pressure in the reservoir increases, the drive is shifted toward its stop. The force sensor disposed between the drive and the pump housing has a bending bar so that the force sensor is deformed in proportion to the reactive force on the bending bar. The elastic O-rings also undergo deformation and counteract the movement of the drive. Since the O-rings have a large circumference, the restoring forces created by the elastic deflection are relatively great so that the measurement result for the pressure in the reservoir is greatly falsified. The two sealing sites in FIG. 1 of DE 19840992 A1 permit only a seal between the displacement platform of the drive and the fixed pump housing of the metering device.

FIG. 24 of the DE 19717107 B4 also shows that the telescoping displacement stages and the driving stage of the spindle device likewise have gaskets to prevent leakage fluid from penetrating into the electronics and/or a power source of the metering device through the spindle device. According to FIG. 24, this requires three additional sealing sites, by means of which the two movable displacement stages and the fixed driving stage can be sealed. Despite the great technical complexity of sealing the drive to protect the electronics and the power source, the disadvantage still remains that the spindle device and/or the spindle thread cannot be protected from soiling. The spindle device of the D-TRONplus pump cannot be protected from soiling, such as, for example, dust, insulin, cleaning agents for cleaning and water. Such soiling can significantly reduce the lifetime and reliability of this spindle device, which is permanently mounted on the housing, because these factors accelerate corrosion and increase the friction in the spindle drives. However, it is advantageous that the drive, which is mounted axially above the force sensor, can detect the spindle device riding up onto the plunger of the reservoir. In this way, the fluid level and/or the amount of insulin in the reservoir can be determined automatically with either partial or complete filling, without any action by the user. To do so, starting from a completely retracted position, the spindle device is moved in the forward direction and the number of motor steps until it runs onto the plunger is determined. The run-up onto the plunger, which causes an increase in force on the force sensor, can be detected by a control unit. If the control unit detects the run-up, then it shuts down the motor. The motor steps carried out up to the run-up are subtracted from the total number of motor steps, and the filling level is finally determined in this way. The D-TRONplus pump is from the first generation of insulin pumps, in which the insulin pump is worn close to the body, for example, in a pants pocket, and the insulin is conveyed through a catheter tube to an infusion site formed by the cannula for dispensing to a user.

A second-generation metering device is disclosed in WO 2006/104806. Second-generation devices are worn directly on the body. They have a bottom side, which is prepared for attachment to the body, and they can be glued directly to the abdomen or the arm, for example. It is advantageous here that no catheter tube is needed between the pump and the injection site, as is the case with the first-generation devices. However, second-generation devices such as the OmniPod insulation pump from Insulet Corp. have the disadvantage that these devices can only be used once. Second-generation devices do not have any components that can be used repeatedly by a user. The spindle device according to WO 2006/104806 has a spindle rod, which has a thread and is fixedly connected to the plunger. The plunger is in its extended position before filling. During the filling, the plunger together with the spindle rod is moved in reverse, wherein a drive wheel that is not locally displaceable releases the spindle rod in the reverse displacement and filling to form a spindle drive. This means that the drive wheel and the spindle rod are not coupled to one another directly in the reverse displacement and the associated filling of the reservoir. The drive wheel which is driven is coupled to the spindle rod via a spindle drive only when dispensing the liquid medication through the advance of the plunger. In filling the reservoir, there is a relative displacement between the locally fixed drive wheel and the spindle rod, which can be displaced in reverse. In the reverse displacement of the spindle rod, the spindle rod abuts against a sensor rod when there is a defined reservoir volume, so that by further displacement of the spindle rod, the sensor rod is deflected transversely to the spindle rod toward a contact point. By contacting the contact point, a contact signal is generated and sent to a control unit. As soon as the control unit recognizes the contact signal, the plunger is in a defined position with a known filling volume. One disadvantage of this new filling level determination method is that it is technically susceptible to problems and is inaccurate due to many geometric tolerances and tolerance chains; also, it can only monitor a minimal filling level. A single sensor unit formed by a sensor rod and a contact point is therefore not sufficient for monitoring the filling level over the entire distance of travel of the plunger. For this purpose, multiple sensor rods and contact points must be disposed along the path of travel of the spindle rod, which makes the filling level determination technically more complex and more expensive.

In addition, the device according to WO 2006/104806 can be used only once, so that the cost of the device for treatment is too high. In addition, the device has no protection against internal reservoir leakage, which reduces the safety level for the user due to short circuits and failure in the electronics and/or the power supply. Medical metering devices may have additional sensor systems for monitoring. In dispensing fluid medications, complete or partial obstructions or closures may occur in the fluid-carrying channel; these are known as occlusions in the technical jargon. Occlusions result in a suboptimal supply of liquid medication to the user which can have a negative influence on the therapeutic result. In insulin pump therapy, occlusions result in an undersupply of insulin to the patient, which leads to an uncontrolled rise in the blood glucose level. The metering devices are monitored for the occurrence of occlusions during operation by means of suitable sensor systems so that these devices are capable of detecting the occlusions automatically and sending an alarm signal to the user. Detection of occlusions, i.e., detection of complete or partial occlusions in the fluid-carrying channel, which may occur due to kinks in a cannula in the fluid-carrying path or growth of tissue into the cannula, for example, is accomplished in WO 2006/104806 by means of a second sensor unit, which may in turn consist of sensor rods and contact points, for example. It is a disadvantage for the device according to WO 2006/104806 that several sensor units are required for occlusion detection and minimum filling level monitoring.

The third-generation devices are those that are worn on the body, like the second-generation devices, but they are different from the devices of the second generation in that they also have a reusable part in addition to the disposable parts. Devices of this latest generation are known from WO 2010/076792 A1 and US 2011/0213329 A1, for example. For both of the embodiments disclosed in WO 2010/076792 A1 and US 2011/0213329 A1, the disposable part comprises a reservoir, a plunger, a spindle device disposed on the disposable part, a power source and a fluid connection from the reservoir to the injection site. Here again, in order to fill the reservoir, the plunger is displaced in reverse from an initially extended position into a filled starting position. Then liquid medication flows from the storage container in the reservoir. The spindle device of US 2011/0213329 A1 comprises a spindle rod rigidly connected to the plunger and a spindle nut, which has a variable diameter and surrounds the spindle rod. During filling, there is a relative displacement between the spindle rod and the spindle nut here, as is also the case for the metering device of the second generation according to WO 2006/104806. Only when the disposable part is connected to the reusable part is the spindle nut coupled to the spindle rod via a threaded connection. When the disposable part is connected to the reusable part, the spindle rod is first brought into engagement with a drive sleeve. In connecting the two housing halves, i.e., in connecting the reusable part to the disposable part, the spindle nut is accommodated by the drive sleeve axially on a seat. The seat, which is formed on the drive sleeve to receive the spindle nut, is designed in a conical shape. In displacement of the spindle nut toward its fixed seat which is designed with a conical shape, the diameter of the spindle nut is reduced until an inside thread of the spindle nut engages in an outside thread of the spindle rod and thereby establishes a fixed spindle drive and/or a fixed spindle connection between the spindle nut and the spindle rod. The spindle nut may be supported directly on the drive sleeve. Alternatively, it may also be supported on a stop formed on the disposable part.

With the device described in US 2011/0213329 A1 both partial and complete fillings of the reservoir are possible. Therefore, residual amounts of insulin, which must not be used by the user, can be prevented. The daily demand—total daily dosage, abbreviated TDD—can vary greatly. Children require approximately 10 to 50 IU insulin per day, whereas adults require between approximately 40 and 150 IU insulin daily. The metering device disclosed in US 2011/0213329 A1 has a sensor unit, which sends an alarm to the user when the filling level drops below a defined specification. To do so, the metering device has a sensor unit formed from a light source and a detector, used to monitor the position of the spindle rod. Here again, it is a disadvantage that said sensor unit can monitor only a minimum filling level. When the filling level drops below the minimum, an alarm can be sent to the user. To make it possible to monitor the filling level more accurately, it is proposed that additional sensor units formed from light sources and detectors be disposed along the longitudinal axis of the drive sleeve. Such a design having a plurality of sensor units disposed along the drive sleeve for monitoring the filling level requires a relatively large amount of space and is more susceptible to problems during operation due to the increased complexity. As an alternative an inductive coil disposed around the drive sleeve is proposed for the filling level determination. This variant requires just as much space and is not optimal for compact metering devices to be worn on the body. The specific embodiments for the filling level determination and the occlusion detection are described in further detail in WO 2009/125398. For the occlusion detection, another sensor unit, which is a second sensor unit, is used, consisting of a flexible connecting tube disposed on the disposable part and a spring-guided sensor plate disposed on the reusable part.

The metering device described in US 2011/0213329 A1, having the sensor units that are described in WO 2009/125398 for the filling level determination and the occlusion detection is complex and expensive, difficult to assembly and also takes up a great deal of space. It is a distance in particular that different sensor units are used for the filling level determination and the occlusion detection. The device disclosed in US 2011/0213329 A1 has other disadvantages. For example, the drive sleeve does not have any sealing locations, so that a leak originating from the reservoir can reach the drive sleeve to the gear and can even advance further into the interior of the reusable part. If liquid medication such as insulin reaches the reusable part, there may be corrosion damage to the components. Such damage usually results in a substantial reduction in lifetime due to accelerated aging of components. In addition, it should be pointed out that it is a disadvantage that the spindle nut is supported on the drive sleeve. The connection formed by snap-hooks between the housing of the disposable part and the housing of the reusable part in general has some axial play. Axial compression of the two housing halves may result in the plunger being displaced relative to its wall and this causes the metering device to dispense a corresponding amount of insulin. Such effects can have a negative influence on the therapeutic result because even tiny relative displacements of the housing halves with respect to one another can result in therapeutically relevant bolus doses. For example, a displacement of 0.01 mm of the housing halves with respect to one another leads to an equivalent displacement of the plunger in the reservoir by 0.01 mm. If the reservoir has a cross section of 125 mm², the result is a bolus dose of 0.125 U insulin when using insulin in the concentration of U100 insulin.

WO 2010/076792 A1 describes another specific embodiment of the spindle device, but in this case the spindle rod is driven instead of the spindle nut. The spindle rod in this specific embodiment has an articulated connection to the plunger and is supported on the plunger supported on the plunger by means of a ball joint. Again in this embodiment variant, the spindle rod and thus the plunger are moved backward from a retracted position to a starting position in order to draw liquid medication from a storage container into the reservoir. The spindle nut is again disposed in a stationary position on the disposable part so that during the filling, the spindle rod is moved in reverse relative to the stationary spindle nut. In this specific embodiment, the spindle rod is driven to rotate, so a stationary spindle nut, which is secured against twisting, is necessary to create the spindle rod advance wherein the spindle rod can be supported on this spindle nut in dispensing the liquid medication. After the filling, the disposable part is connected to the reusable part. In doing so the spindle rod is inserted into a drive sleeve disposed on the reusable part. In combining the two housing halves, the spindle nut is brought further into engagement with the spindle rod by means of a thread connection.

The exemplary embodiment in WO 2010/076792 A1 which differs from the exemplary embodiment described above only in that the spindle rod is drive to rotate instead of the spindle nut, does not eliminate any essential weaknesses or disadvantages of the previous specific embodiment described in US 2011/0213329 A1. Again the same sensor units and devices as those described in WO 2009/125398 are used for the filling level determination and the occlusion detection. With both embodiment variants described in US 2011/0213329 A1 and WO 2010/076792 A1 it is also difficult to bring the spindle rod of the disposable part into engagement with the drive sleeve of the reusable part. Especially for diabetics whose motor abilities may be limited, no guides or aids are provided for a coupling of the two housing halves that would be easier to handle. Nor is any seal provided for the drive sleeve even in WO 2010/076792 A1.

WO 2008/024814 A2 discloses a metering device having a reusable part and a disposable part, as illustrated in FIG. 18 through FIG. 22. This exemplary embodiment is also one of the third generation metering devices. The exemplary embodiment illustrated in FIG. 18 through FIG. 22 shows spindle rod passing through the plunger and into the reservoir, wherein the plunger itself is designed as a non-rotating spindle nut. A coupling member for transferring the drive power to the spindle device of the disposable part is provided on the reusable part. The coupling member is additionally connected to an output wheel of the gear, wherein a sealing location is provided between the coupling member and the output wheel, and the coupling member has a stop for the spindle rod. The exemplary embodiment shown in FIG. 18 through FIG. 22 of WO 2008/024814 A2 is advantageous with regard to the sealing of the reusable part. The sealing location is formed on a slender rotating driven shaft. In the technical implementation, however, it is difficult to provide a seal on a rotating spindle rod on a plunger. In practice, such a spindle rod mounted to pass through the plunger has not yet been implemented in an acceptable working product available on the market. Furthermore, it is difficult to fill the reservoir. Since the spindle rod is disposed in the interior of the reservoir, joint displacement of the plunger together with the spindle rod mounted on the plunger is impossible. It is therefore more practical for filling purposes to displace the plunger by rotation of the spindle rod in order to thereby draw liquid medication from a storage container into the reservoir. By means of this method, in which the plunger is advanced in reverse from a forward position for the transfer of liquid medication, both partial and complete filling are conceivable. The spindle nut disposed on the plunger is thereby moved again relative to the fixed spindle rod, as is also the case for the examples of the second and third generation already mentioned above.

The following table summarizes the state of the art, comparing metering devices of the first generation, the second generation and the latest, the third generation, and also comparing them by the important criteria of reliability and longevity, handling and cost. An important criterion that increases reliability is the arrangement of the spindle device. The spindle device is disposed on the reusable part in first-generation devices and therefore it cannot be replaced. It is advantageous if the spindle device is disposed on the disposable part and then is replaced with each new disposable part. Furthermore, the lifetime of the metering device can be improved if the reusable part to prevent internal leakage from the reservoir. This ensures, for example, that electronic components, the gear and the motor of the reusable part do not come in contact with liquid medication, which could lead to accelerated aging of the metering device. One criterion for improving the handling is an automatic filling level determination, which facilitates handling for the user and eliminates dosing errors, which may occur with manual input of the filling level by the user. It is also particularly advantageous if the same sensor system that is used for the filling level determination can also be used for pressure monitoring of the reservoir. Manufacturing costs can therefore be lowered and the complexity of the device can be reduced. The costs incurred in treatment are another important criterion in evaluating and differentiating metering devices. An innovative metering device should not cause extremely high costs in use. Second-generation metering devices do not usually have any reusable components and are discarded as a whole after a single use. Third-generation devices have been improved to the extent that they have disposable parts but they also have one reusable part and can achieve a cost advantage in this way.

The following table shows that no generation of the state of the art is capable of fulfilling all the criteria, such as reliability and lifetime, handling and costs at the same time. At most three criteria are covered and met of the five criteria defined here. Some metering devices can fulfill only two of the five criteria simultaneously.

| | | Lifetime and | | | | |
|---|---|---|---|---|---|---|
| | | reliability | | Handling and safety | | |
| | | Spindle | Seal to | | Same | |
| | | device on | prevent | Automatic | sensors for | Costs |
| | | disposable | internal | filling level | pressure | Semi- |
| | Criterion | part | leakage | determination | monitoring | disposable |
| 1^{st} | D-TRON - | No | No - | Yes - via | Yes - via | Yes |
| gen. | Roche | | spindle | force sensor | force sensor | |
| | DE 19717107 | | device | | but | |
| | B4 | | cannot | | inaccurate | |
| | DE 19840992 | | be | | measurement | |
| | A1 | | sealed | | | |
| 2^{nd} | OmniPod - | Yes | No | No, only | No, different | No |
| Gen. | Insulet | | | monitoring of | sensors | |
| | WO | | | minimal | | |
| | 2006/104806 | | | filling level | | |
| | A2 | | | | | |
| 3^{rd} | Medtronic | Yes | Yes | No | No, different | Yes |
| Gen. | EP 2407192 | | | | sensors | |
| | B1 | | | | | |
| | WO | | | | | |
| | 2008/024814 | | | | | |
| | A2 | | | | | |
| 3^{rd} | Solo M - | Yes | No | No, only | No, different | Yes |
| Gen. | Roche | | | monitoring of | sensors | |
| | WO | | | minimal | | |
| | 2010/076792 | | | filling level | | |
| | A1 | | | | | |
| 3^{rd} | Solo M - | Yes | No | No, only | No, different | Yes |
| Gen. | Roche | | | monitoring of | sensors | |
| | US | | | minimal | | |
| | 2011/0213329 | | | filling level | | |
| | A1 | | | | | |

Second-generation devices have a lifetime of only a few days, usually approximately three days of use in continuous infusion of insulin, hereinafter referred to as CSII—continuous subcutaneous insulin infusion. Such devices are discarded as a whole, so that the treatment costs unfortunately turn out to be high and due to the high cost, not all users can gain access to this technology, which is a significant disadvantage. First-generation metering devices have the disadvantage that a noticeable catheter tube connection exists between the metering device and the user; it cannot be worn discretely and interferes with everyday activities. With the first-generation metering devices, the spindle device is fixedly disposed on the reusable part and therefore exposed to constant wear. In the case of the D-TRONplus pump, which is described in the specifications of DE 19717107 B4 and DE 19840992 A1, the threads of the spindle device are exposed to high wear specifically due to soiling because there is no seal. High wear on the spindle device can greatly shorten the lifetime and reliability of an insulin pump. However, the automatic filling level determination and pressure monitoring of the reservoir by means of the force sensor are advantages.

However, devices of the third generation have been improved to the extent that the spindle device, as the essential wear part, can be moved from the reusable part to the disposable part in the design stage, to thereby improve the reliability of the metering device and increase its lifetime. These devices do not have automatic filling level determination by means of a force sensor, for example, which is another important disadvantage of the third-generation devices. In devices of the second and third generations, a spindle rod that is connected to the plunger fixedly or via an articulated connection is displaced in reverse relative to a spindle nut during the filling of the reservoir. The relative displacement between the spindle rod and the spindle nut, which can be carried out by the user in filling, has the unfortunate result that additional sensor systems are required to determine the position of the spindle rod with respect to an absolute coordinate system and ultimately to be able to monitor a minimum filling level on this basis. The second-generation device discussed above has the sensor unit formed by the sensor rod and a contact point, as described above for this purpose. Third-generation devices have an alternative sensor solution for this purpose, consisting of a light source and a detector for the light source. This alternative can also monitor only a minimum filling level, which is a disadvantage, and it is impossible to determine the effective filling level. This would require additional sensors. In order for the third-generation metering devices to have the shortest possible length, the axial extent of the plunger is designed to be as short as possible. This allows the formation of a compact metering device, but on the other hand, it is a disadvantage because the plunger held in the reservoir is not guided well and tumbling of the plunger may occur as it advances, and this is in turn associated with a reduced accuracy in dispensing. One measure of the quality of the bearing of the plunger in a reservoir to prevent tumbling is the length ratio L/Do, where L is a longitudinal axis of the oval plunger cross section and Do is the distance between sealing sites on the plunger. Both the second-generation devices and the third-generation devices have unfavorably large length ratios here of more than 2.5 due to the fact that they have a rigid or articulated connection of the spindle rod and plunger. The L/Do ratio advantageously has a value of more than 0.5 and less than 2.5.

In addition, telescoping single-stage spindle devices are known from WO 94/15660 and WO 97/00091. The telescoping spindle devices are always designed with the same arrangement and have first and second displacement stages, wherein the first displacement stage receives the second displacement stage, enclosing it, and the second displacement stage receives a driving stage, enclosing it. With this arrangement, the driving stage drives one of the two displacement stages, so that the displacement stages are extracted sequentially. The telescoping spindle devices of WO 94/15660 and WO 97/00091 are supported on a fixed supporting part, which also serves as a twist-lock base for the displacement stages. Likewise, the driving stage is mounted on the supporting part in a manner that is resistant to axial displacement. The supporting part and a reservoir body can both be connected to an intermediate part. The releasable connection between the supporting part and the intermediate part is established by means of a magnetic connection. The intermediate part and the reservoir connected to it can also be connected to a reusable driving device. The intermediate part and the reservoir connected to it can additionally be connected to a reusable driving device. In the coupled state, a laterally driven driving gearwheel of the driving device drives an axially positioned driven gearwheel of the spindle device. The driven gearwheel is rigidly connected to the driving stage. The driving stage and/or the spindle device, respectively, is not supported on a fixed solid housing but instead is supported axially on the support part.

The telescoping devices of WO 94/15660 and WO 97/00091 are complex in their design, require many parts, have a complicated assembly and are difficult to handle. Furthermore, it is always necessary to fill the reservoir entirely because only then can the driven gearwheel of the spindle device be coupled to the driving gearwheel of the driving device. In addition, the embodiments according to WO 94/15660 and WO 97/00091 do not have any means for protecting the driving device from internal leakage. WO 94/15660 also discloses a one-stage spindle device having a spindle rod disposed so that it passes through the plunger. The spindle rod is supported on an intermediate part. The intermediate part here supports the spindle rod axially in a displacement-proof manner, holds a reservoir body and can be connected to a fixed housing of a driving device. Forces acting axially are transferred via the spindle device to the intermediate part and are absorbed by the latter. Here again, the driving device has a coupling element for the spindle rod. When filling the reservoir, the plunger on which the spindle nut is formed is moved in reverse and relative to the spindle rod. Here again, there are no means for protecting the driving device from soiling, leakage of the reservoir and other liquid media.

### SUMMARY

One aspect of the present invention relates to a metering device for dispensing the liquid medication, in particular for dispensing insulin, which will overcome the disadvantages described here of the first, second and third generations and in particular to improve upon a metering device with regard to such criteria as lifetime and reliability, manufacturing costs and costs of treatment as well as handling for the user.

The metering device has one reusable part and at least one disposable part for administering liquid medication to a user's body, wherein the liquid medication can be dispensed in a metered form for the purpose of an infusion or an injection from a reservoir by advancement of a plunger held in the reservoir. The reusable part comprises a housing, a control unit and a driving device, wherein the driving device has a coupling element for transfer of a drive power to a spindle device for advancement of the plunger, and the coupling element is fixedly connected to an output wheel of the driving device and has an axial stop for the spindle device. In addition, a sealing location may preferably be disposed between the output wheel and the coupling element. The at least one disposable part comprises the reservoir and the plunger that is held in the reservoir and can be advanced by the spindle device, wherein the spindle device is disposed on the disposable part and has at least one spindle drive, which is formed in particular by an inside thread and has a distance of travel (V) per spindle drive, wherein the spindle device (S) of the disposable part (1) can be coupled to the reusable part (2) via the coupling element. In addition, the metering device comprises a fluid-carrying connection between the reservoir and the user and a force sensor disposed on the reusable part, measuring the reactive force of the spindle device, which serves as a measure of the pressure in the reservoir. Due to the fact that:
i) the metering device is designed so that the spindle device (S) of the disposable part (1) is in the retracted state after a partial or complete filling of the reservoir (A) and thus a defined always constant total stroke (H) of the spindle device (S) can be traveled,
ii) the coupling element is slidingly supported axially and the output wheel (14) is supported on a fixed base via the force sensor (15), wherein because of the fixed connection between the coupling element and the output wheel, the reactive force of the spindle drive (S) acting axially on the stop (12) is transferred to the force sensor (15) via the output wheel when the spindle device (S) comes to rest against the stop (12),
   it is possible to form a metering device, which can be improved significantly in comparison with the state of the art.

It has advantageously been found that the present invention improves upon a metering device of a third generation such that the filling level determination and the occlusion detection can take place via the force sensor, such as that known from the D-TRONplus pump of the first generation, while at the same time, the advantages of the third-generation devices, such as reliability, good sealability and replaceability of the spindle device have been retained or even improved.

In addition, it has advantageously been found that the metering device according to the invention, which may be formed from at least one disposable part with a spindle device and one reusable part, can use the same sensor system in the form of a force sensor for occlusion detection and filling level determination, the lifetime and reliability of these sensors being improved by shifting the disposable parts, such as the spindle device, from the reusable part to the at least one disposable part; these are simple to manufacture and lead to reduced costs for the cost carrier in treatment.

The improvements in comparison with the state of the art are listed below, because significant advances are achieved by combining features i) and ii):
Combining features i) and ii) achieves the result that both the filling level determination and the occlusion monitoring can take place via the force sensor, wherein the spindle device according to the invention is disposed on at least one disposable part. After the coupling, the spindle device can move only toward the stop and opposite the driving device. Since the plunger is held in the reservoir by means of friction, a reactive force is generated when the spindle device is run up onto the stop formed on the coupling element. This reactive force is transferred to the force sensor via the axially supported coupling element together with the output wheel. If the reactive force achieves a defined target level, the control unit can detect the run-up of the spindle device onto the stop and can shut down the drive unit. The drive unit may have a gear reduction in the form of a gear and/or driving means in the form of a motor, a memory alloy or some other driving means. According to the invention, the coupling element, by means of which the spindle device is driven, is disposed at the output of the drive unit. In general the drive unit is moved in discrete increments, wherein one increment may correspond to a constant angle increment. The number of steps until the spindle device runs up onto the stop can be used by the control unit to calculate the filling level. For each new reservoir, depending on whether it is partially or completely filled, the drive unit always moves a constant number of steps because according to the invention the spindle device is in its retracted state after filling. The number of steps available for dispensing the liquid medication after running up against the stop is proportional to the initial filling level. By adding up the steps in dispensing the liquid medication, an instantaneous filling level of the reservoir can also be calculated.
In dispensing the liquid medication, the controller monitors the occurrence of occlusions in the fluid path based on the measurement signal of the same force sensor as for the fluid level determination; such an occlusion may occur due to kinks in the cannula or growth of tissue into the cannula. If an occlusion develops in the fluid-carrying connection, the pressure in the reservoir will increase steadily during further dispensing of liquid medication. For example, if a threshold is exceeded, the controller can send an alarm to the user regarding an occlusion. A wide variety of algorithms can be used for evaluation of the measurement signal of the force sensor. In the simplest case, an occlusion alarm is sent as soon as a constant force value or threshold is exceeded. However, algorithms with which a series of measured values is used over a certain interval of time is also conceivable. In addition to the force values, which are determined by means of the force sensor and can be stored in a controller, other signals such as, for example, an engine current in dispensing the liquid medication may also be taken into account in the algorithms. Due to the rising pressure in the reservoir when there is an occlusion, the motor currents also increase due to the increasing load on the motor.
According to the invention the spindle device is disposed on at least one disposable part. This achieves the result that a new spindle device is used for each new reservoir. The reliability and the lifetime of the metering device according to the invention can be improved in this way because the spindle device always has a constant drive torque in delivery of fluid because it is always replaced and therefore is not exposed to any wear. In the state-of-the-art D-TRONplus pump, in which the spindle device is disposed on the reusable part, the torque of the spindle device increases with the lifetime of the device due to wear and friction, which leads to a reduction in reliability and lifetime.
The metering device according to the invention also facilitates handling for the user because it is not necessary to rotate the spindle device back to the starting state. With the D-TRONplus pump, rotation of the spindle device back to the starting state can take several minutes and requires a great deal of energy.
In addition, according to the invention, the force sensor is disposed directly beneath the output wheel. Therefore, the reactive force acting on the stop, corresponding to the force of impact of the spindle device can be measured with greater accuracy than is possible with the state-of-the-art designs. The reactive force acting on the stop is transferred via the output wheel to the force sensor. The sealing location between the output wheel and the coupling element influences the force acting on the force sensor and can also influence the measurement result of the force sensor. The basic goal is to measure the pressure in the reservoir by means of the force sensor with the greatest possible accuracy. The pressure in the reservoir exerts a compressive force on the plunger, and the frictional force acting between the plunger and reservoir also acts in addition to the compressive force on the spindle device. The impact force acting on the spindle device is transferred to the output wheel via the stop of the coupling element and the output wheel is held via the force sensor. The reactive force introduced axially on the force sensor is therefore proportional to the compressive force in the reservoir, wherein it can be influenced by the frictional force on the plunger and the frictional force of the sealing site between the output wheel and the coupling element. The goal is to minimize the frictional force on the sealing site. This can be achieved by connecting the output wheel and the coupling element to one another by means of a slender shaft. The slender shaft permits a good seal, and furthermore, the frictional forces of the sealing site are low so that the accuracy of the measurement of the compressive force by means of the force sensor can be improved.

Metering devices of the first generation are a disadvantage because they cannot be worn discretely due to the catheter tubing and they can restrict the freedom of movement of the user. Furthermore, the spindle device is disposed permanently on the reusable part and therefore is exposed to constant wear during operation, which can limit the lifetime of the metering device. However, the D-TRONplus pump of the first generation offers the advantages that occlusion detection and filling level determination can take place by means of the same sensor system, i.e., by means of the force sensor. Therefore the complexity and manufacturing costs of the metering device can be reduced. In devices of the second and third generations, a spindle rod is always moved in relation to a spindle nut during filling so that a filling level determination cannot be performed by using a force sensor since the position of the spindle rod relative to the spindle nut is not known after filling. With all of the second- and third-generation devices the spindle device is not disposed on the reusable part but instead is on the disposable part in order to improve the reliability of the metering device in this way. In the filling, the spindle rod is always displaced relative to a spindle nut. This relative displacement causes a loss of information with regard to the position of the spindle rod relative to the spindle nut because after filling, the position of the spindle rod relative to the spindle nut is unknown. In the second- and third-generation devices, after the disposable part has been connected to the reusable part, the position of the spindle rod is determined in relation to a stationary sensor system, which is disposed on the reusable part. Therefore, at least a minimum filling level can be monitored. More complex sensor systems here allow maximum monitoring of the filling level along the spindle rod, which is a disadvantage. Therefore, second- and third-generation devices require two separate sensor units for occlusion detection and filling level determination, but that drives the costs and increases the complexity of the pump. The filling level determination and/or the minimum filling level monitoring is/are always determined by monitoring the spindle rod position.

The invention here has recognized the fact that the spindle device should be disposed on at least one disposable part as is the case with the systems of the second and third generation, in order to form the most reliable possible and long-lived metering device. In contrast with the second- and third-generation devices, the present invention has also recognized that the spindle device must be in the retracted state after a partial or complete filling and in this retracted state the position of the spindle rod relative to the spindle nut of the spindle drive, for example, is known. It is therefore possible to achieve the result that, after filling, the spindle device can be moved in a total stroke that is always the same. This is true of spindle devices having a spindle drive but is also true of telescopic spindle devices having at least two spindle drives. The fact that the spindle device is in its known retracted position after filling makes it possible to overcome a significant disadvantage of the second- and third-generation devices, in which the position of the spindle device after filling is unknown. This principle, according to which the position of the spindle device after filling is defined and known, is utilized in the present invention. This finding also allows the filling level determination, in addition to the occlusion detection, to take place by way of the force sensor for the metering device according to the invention. In contrast with the D-TRONplus pump, the spindle device does not move from a retracted position only in the forward direction but instead is moved in reverse, i.e., opposite the forward direction against the stop and then in the forward direction for dispensing the liquid medication, so that it always travels the distance of a total stroke is always executed, regardless of the initial filling level.

With the metering device according to the invention, the force sensor is disposed directly beneath the output wheel. The output wheel is slidingly supported against the force sensor. With the first generation D-TRONplus pump, the force sensor is disposed between the displacement platform and the pump housing wherein the displacement platform is held in the pump housing by means of O-rings.

The invention has also recognized that it is particularly advantageous not to arrange the force sensor beneath a displacement platform but instead to arrange it directly beneath the output wheel of the driving device. The output wheel and the coupling element may be connected to one another via a shaft. A sealing site may preferably be disposed on the shaft. It may be formed by O-rings but it can also be implemented by an accurate guidance on the housing without using O-rings. In the latter case, the seal is provided only by the guidance and bearing of the shaft on the housing, which is designed to be tight. The shaft is designed to be slender in general. In addition, it is driven to rotate for transfer of the drive power to the spindle device. In the case of a seal formed by an O-ring, only minor axial frictional forces occur due to the slender design of the shaft and the rotational movement of the shaft. The slender shaft therefore allows a better seal in comparison with the first-generation D-TRONplus pump. In addition, the metering device according to the invention can perform measurements more accurately of the pressure prevailing in the reservoir in comparison with the D-TRONplus pump of the first generation. Occlusions can thus be detected more rapidly and more reliably, which results in a definite therapeutic improvement. Due to the more rapid detection, the volume built up in the reservoir in the event of an occlusion, which is referred to in the technical jargon as occlusion volume or occlusion bolus, is also greatly reduced. The faster detection of occlusions as well as the reduction in the corresponding occlusion volumes can significantly improve the treatment and thus also control of the blood glucose levels.

It is the accomplishment of the invention to combine the technical features i) and ii), so that all of the five criteria defined previously in the state of the art can be met. These criteria will be listed here again and the improvements in comparison with the state of the art will be discussed. The metering device according to the invention should be designed so that it is reliable and long-lived. This is achieved by disposing the spindle device on at least one disposable part and preferably designing the reusable part, so that it can be sealed well with respect to the outside. In addition, the filling level determination and the occlusion detection should take place by way of the same sensor system, so that the reliability and long-lived nature of the metering device according to the invention can be improved while manufacturing costs can be reduced. This is achieved by arranging a force sensor directly beneath the output wheel of the driving device for this purpose and having the spindle device always in the retracted state after filling. In this way the filling level determination can be carried out automatically by means of the force sensor. Furthermore, a slender shaft, which is especially well-suited for sealing the coupling element, may be disposed between the output wheel and the coupling element. The pressure in the reservoir can be determined with greater accuracy due to the reduced frictional forces on the sealing site, so that occlusions can be detected more rapidly and more reliably. In addition, the metering device according to the invention should be capable of holding and ejecting partially filled reservoirs. To reduce the cost of treatment, the metering device according to the invention should be formed from disposable parts and one reusable part.

The metering device according to the invention may preferably be designed in such a way that after coupling between a recently-filled disposable part and the reusable part, the spindle can be extended out toward the stop of the coupling element to eliminate any longitudinal play in the direction opposite the direction of advance and/or to be extendable when the spindle device comes to rest against the stop in the forward direction. It is basically conceivable that there is no longitudinal play in the coupling between a recently-filled reservoir and the reusable part. In this case the spindle device is already abutting against the stop in coupling and can be extended away from this only in the forward direction. In this specific case, the reservoir is completely full and the spindle device moves only in the forward direction.

Advantageous embodiments of the invention are also disclosed.

The coupling element is advantageously designed as a profiled driving rod with the stop for the spindle device. It is particularly advantageous when in the coupling between the at least one partially or completely filled disposable part and the reusable part, the driving rod of the reusable part can be inserted into an axial elongated hole in the spindle device of the replaceable part and by extracting the driving rod from the axial longitudinal hole of the reusable part and can be uncoupled from the reusable part by extracting the driving rod from the axial longitudinal hole of the disposable part. The driving rod as a coupling element can be cleaned to remove impurities particularly well. The spindle drives of the spindle devices are formed from spindle nuts and spindle rods in general wherein the spindle nut may have an inside thread and the spindle rod may have an outside thread, for example, in the case of a one-step spindle device. The element of the spindle device that is driven by the driving device is referred to as a driving stage and may be, for example, a spindle nut or a spindle rod. The movable element of the spindle device is referred to as a displacement stage and may be, for example, a spindle nut or a spindle rod. By definition, the elongated hole is formed on the driving stage so that in coupling, the driving rod of the driving device is inserted into the longitudinal hole of the driving stage of the spindle device. The driving stage is thus driven to rotate and may or may not be axially movable. For example, a spindle nut may be driven to rotate and a corresponding spindle rod of a one-step spindle device may be extendable, so that the spindle nut serves as a driving stage but is not axially displaceable. It is also conceivable for the spindle nut to be disposed so that it axially and rotationally fixed, such that the corresponding spindle rod can be rotationally driven and can thus be designed as a driving stage and is also extendable.

After coupling with a recently-filled disposable part, the driving stage is connected to the driving rod in a rotationally fixed manner. In rotation of the driving rod by the drive unit, the driving stage is driven to rotate. The driving stage moves axially in the opposite direction from the forward direction and thus comes to a rest against the stop relative to the driving rod up to the spindle device. After running up onto the stop, the spindle device moves in the forward direction. The spindle device of the metering device according to the invention may have a spindle drive and may thus be designed in one stage but it may also be designed to be telescoping and may include two or more spindle drives in order to form a particularly compact metering device in this way. For a telescopic spindle device having two spindle drives, the driving stage may be formed from two sleeves which may be connected to one another on one end side, preferably on the end sides facing the plunger. For example, an outside thread and an inside thread may be provided on the outer sleeve. The inner sleeve may have the elongated hole for the driving rod. The outside thread may be engaged with an inside thread of a first displacement stage and the inside thread may be engaged with an outside thread of a second displacement stage.

Since the spindle device is in a retracted state after filling according to the invention, the driving rod must have a defined length so that a partially filled reservoir can be coupled by means of the driving rod. It is advantageous that the length of the driving rod is the same as or greater than a distance of travel V of a spindle drive. This makes it possible to be sure that partially filled reservoirs can be coupled to the reusable part and can be dispensed. For example, if a spindle device consists only of a spindle drive with a distance of travel V, then initial filling levels of 0% to 100% are allowed for the reservoir. However, if the spindle device has a telescoping spindle with two spindle drives each with a distance of travel V, then filling levels of 50% to 100% are admissible and dispensable with a driving rod with a length V. The length of the driving rod thus depends on the minimum filling level desired initially wherein the number of spindle stages of the spindle device also has a direct influence on the minimum filling level of the reservoir. The minimum filling level may assume general values between 0% and 100% for one-stage spindle devices, and the driving rod is to be adapted to the desired minimum filling level. For a reservoir with a minimum initial filling, the spindle device must still be couplable to the driving rod during coupling.

The length of the driving rod can be determined from this specification. The length of the driving rod thus defines the minimum filling level which can be dispensed by the metering device. Minimum filling levels of 50% to 100% are desirable in insulin therapy so that each user will have available the optimum amount of liquid medication for three days of use, for example. The driving rod of the metering device is inserted into the elongated hole in the driving stage at the time of coupling. The elongated hole may be formed on a spindle rod or a spindle nut. The elongated hole is preferably formed on the spindle rod so that the spindle rod is driven to rotate during this delivery. In this case the spindle nut is designed to be rotationally fixed. Of course here again a reversal is also conceivable in which the spindle nut has an elongated hole into which the coupling element of driving device can be inserted. In this case the spindle nut is driven to rotate and the spindle rod is designed to be rotationally fixed. The coupling element may be designed as a fork, for example, whose tines can run parallel to the axis of the spindle device. The spindle rod is preferably designed to be concentric with the output wheel and the spindle device, so that all the components, i.e., the force sensor, the output wheel, the driving rod, the spindle device, the plunger and the reservoir lie on an axis. In general, application cases in which the metering devices are always loaded with completely filled reservoirs are conceivable. With such application cases it is sufficient to design the driving rod as a short coupling element.

Fundamentally, the plunger itself can function as a displacement stage, in which case the plunger may then have a spindle rod fixedly connected to the plunger or may have a spindle nut designed to be stationary on the plunger. A design of the plunger with a spindle nut, wherein the spindle nut may be part of the plunger wall, is advantageous. This allows a flatter metering device to be formed than is the case with the embodiment variant having a spindle rod fixedly disposed on the plunger. Furthermore, it is desirable if the spindle drive has the largest possible diameter because the tendency of the spindle device to develop kinks can be reduced in this way. Spindle drives having a small diameter have a greater tendency to develop kinks than do spindle drives having a larger diameter. To form the flattest possible plunger and to minimize the tendency of the spindle device to form kinks, it is advantageous if the plunger, as a displacement stage, has an inside thread which may be part of the plunger wall. The plunger with its inside thread thus serves as a spindle nut and has a large diameter, which is favorable for the spindle drive. Due to the reduced tendency to form kinks, the plunger can be operated with greater precision in the output of liquid medication because tumbling of the plunger can be reduced in this way. In the reversed variant, in which the spindle rod is disposed fixedly on the plunger, there are disadvantages with regard to the size of the plunger as well as with regard to the tendency of the spindle drive to develop kinks. Therefore an embodiment of the plunger as a spindle nut offers advantages in terms of size, reduction in the tendency to form kinks for the spindle device and a reduction in tumbling of the plunger. An especially advantageous structure of the spindle device with a central driving rod is one which can be inserted into a central longitudinal hole in a driving stage and the driving stage can be connected to at least the spindle nut disposed on the plunger. The plunger here serves as the first displacement stage. The driving stage can be connected to the second displacement stage by means of a second threaded connection so that a telescoping spindle device can be formed. Said arrangement is superior to other arrangements with regard to size for forming flat metering devices that can be worn on the body and it has the best properties with regard to its resistance to kinking.

The driving device has the same direction of rotation for the extension in the direction opposite the forward direction and the extension directed in the forward direction. The complexity of the metering device can therefore be simplified and costs can be reduced. For example, the controller of the driving device can be simplified because the driving device is always operated in only one direction. The first-generation devices require software and hardware to enable motor operation in both directions of rotation because the spindle device, which is permanently disposed on the reusable part, must always be retracted again back into its starting position. Retraction of the spindle device into the retracted state is omitted for the metering device according to the invention because the spindle device is disposed on the at least one disposable part. This makes it possible to save on the power consumption in retraction. The spindle device is preferably disposed on the plunger. The plunger can be displaced together with the spindle device. The user couples the plunger to a pull-up rod for filling the reservoir with liquid medication. Due to the fact that the spindle device is integrated into the plunger, the user can perform the filling in the way in which he is accustomed to from the devices of the first generation the way in which it is familiar to him With this method, the plunger which is in the reservoir is shifted in both directions for filling by means of a pull-up rod. Displacement of a spindle rod relative to a spindle nut, as proposed in the state of the art for the second and third generations, is an action that the user is not accustomed to but this can be avoided by the metering device according to the invention. Users prefer a handling which is self-evident to them and with which they are familiar so they can displace the plunger in the reservoir preferably by means of the pull-up rod. The spindle device may be integrated into the plunger in such a way that the user perceives only the plunger but not the spindle device.

The spindle device is always extendable over a constant total stroke H for each new reservoir, regardless of whether the reservoir is partially filled or completely filled. The total stroke H thus consists of a reverse stroke H1 for eliminating the longitudinal play and a stroke H2 directed in the forward direction for administering the liquid medication. In addition, it is advantageous if the driving device includes a motor and a gear driven by the motor wherein a last gearwheel may be designed as the output wheel. Electric motors, which can be moved in discrete motor steps, are preferably used. In run-up of the spindle device onto the stop formed on the driving rod, a reactive force can be measured by the force sensor and sent to the control unit, and the motor of the drive unit can be shut down by the control unit in this way. The number of motor steps until the run-up onto the stop is subtracted from the total number of motor steps for the total stroke H and a permanent number of motor steps for dispensing the liquid medication is determined in this way. Furthermore, the remaining number of motor steps can be multiplied times a proportionality constant and the amount of liquid medication in the reservoir can be determined from this. The motor that is used may be a brushless dc motor, for example, which can be controlled by means of Hall sensors. The acknowledgment of a rotor position can take place via three Hall sensors offset by 120 angular degrees. Hall sensors offset by 120 angular degrees supply six different switching combinations per revolution. The motor may comprise partial windings which can be energized in six different conductive phases according to the sensor information so that the motor has six motor steps per revolution. The current and voltage curves may be block shaped. The controller monitors the motor steps by means of the Hall sensors. The controller can also count the motor steps. The three Hall sensors make it possible to detect a revolution of the motor in six discrete motor steps of 60 angular degrees each. However, the rotor position of the motor can also be determined by means of a flywheel disposed on the rotor. The flywheel can interrupt a beam of light emitted by a light source or may allow it to pass through to a detector. The control unit can determine the position of the rotor from the beam of light, which is continuous and interrupted in alternation. Additional driving means for the drive unit are known from the state of the art such as memory alloys, for example. The driving means have in common the fact that they have extremely small angle increments, which are proportional to the extremely small metering increments of the metering devices. The smallest metering increment or the metering resolution of the metering device is a characteristic variable for each metering device.

In addition, it is especially advantageous if the housing is formed from three housing parts, wherein a first housing part holds at least the control unit and/or a power source, a second housing part is designed as a chassis and accommodates at least the motor, the transmission and the coupling element designed as a driving rod and a third housing part is designed as the transmission bottom. Basically the force sensor may be supported on the gear bottom. However, it is more advantageous to form the base for the force sensor by means of a plate that can be attached to the chassis. In this way it is possible to achieve the result that the motor, the gear and the coupling element in the form of a driving rod as well as the force sensor may be disposed directly on the chassis. Therefore a particularly compact unit on which all the essential components can be accommodated may be formed. Furthermore, the reactive force on the spindle device is not directed at the gear bottom but instead is absorbed by the plate that can be attached to the chassis. The plate may be made of steel, for example, and can be fastened to two guides that are formed on the chassis and run transversely to the forward direction. The force sensor may be supported directly on the plate. It is particularly advantageous to divide the housing into three housing parts because this allows premounting of the respective components of the metering device on each housing part. After premounting, the three housing parts are connected to one another. In general the driving rod is rigidly connected to the output wheel, wherein an O-ring may serve as the sealing element and it may be disposed on a shaft between the output wheel and the driving rod. In assembly, the O-ring is mounted first on its sealing site formed on the shaft. The unit formed by the output wheel, the shaft, the O-ring and the driving rod is subsequently retracted from a bottom side into the chassis. Finally, the force sensor may be placed beneath the output wheel of the gear and the steel plate is brought into engagement with its guide formed on the chassis to secure the coupling element and the force sensor. In a first assembly step, the entire chassis together with the components that can be attached to the chassis is premounted. The completely mounted chassis unit may comprise the chassis, the coupling element formed from the output wheel and the driving rod, the force sensor, the steel plate, gearwheels of the gear and a motor with Hall sensors, for example, for the triggering. In addition, it is advantageous if the chassis can accommodate the first and third housing parts. Again the battery and the controller may be premounted on the first housing part in advance.

The metering device preferably consists of three housing components. Components of the metering device can be premounted by premounting on the first and second housing parts. Moreover, the gear bottom does not absorb any reactive forces of the spindle device so that the metering device can be further improved. Accommodation of the housing receptacle of the gear bottom on the chassis is force-free due to this design and therefore the longevity of this connection can be increased. Accommodation of the housing of the first housing part on the chassis is also free of internal forces. Said arrangement of the housing parts permits accommodation of the housing parts on the chassis in a manner that allows good connection, free of forces and with good sealing.

It is advantageous to join the at least one disposable part and the reusable part to one another by means of a bayonet connection. It is especially advantageous to design the bayonet connection on the chassis. In addition, the bayonet connection may be disposed approximately at the level of the stop for the spindle device with respect to an axial axis of movement for the spindle device. In order for the user to be able to connect the at least one disposable part to the reusable part more easily and more reliably, the housing may have an axial longitudinal guide for the at least one disposable part by means of which the spindle device can be coupled to the coupling element of the reusable part and by means of which the at least one disposable part can be supplied to a depository for the bayonet connection in a guided manner. The axial longitudinal guide is advantageously formed by two grooves directed toward one another on the housing wherein the disposable part has two cams each engaging in a groove. The longitudinal guide formed on the housing to receive the at least one disposable part is very advantageous. In one step the user connects the at least one disposable part to the reusable part by inserting the cam of the disposable part that protrudes radially outward on the longitudinal guide formed on the housing. The disposable part is thereby secured radially on the housing and is only displaceable axially. By a displacement along the guide toward the depository it comes to the insertion of the driving rod on the longitudinal hole so that the driving device is coupled to the spindle device for transfer of the drive power. While additional displacement of the at least one disposable part, ultimately the cams are guided toward the depository which is designed as a bayonet connection. The depository is formed on the housing, preferably being formed directly on the chassis. The cams are preferably displaced up to an axial stop formed on the chassis and then the cams are rotated about the longitudinal axis in the bearing slot in the bayonet connection formed on the housing by rotation of the at least one disposable part. The longitudinal guide for the cams and the bearing slots are connected to one another. The disposable part can be moved in the slots in a guided manner, which simplifies handling. The bearing slots of the bayonet connection are designed so that the cams can be inserted into the slots so that they fit accurately. The at least one disposable part is held on the housing in the forward direction and also in the direction opposite the forward direction. This type of bearing by means of a bayonet connection in which the at least one disposable part is connected to the reusable part directly on the housing, preferably on the chassis is especially advantageous. For example, it is possible in this way to prevent the at least one disposable part from being displaceable relative to the chassis due to an external force. In the state of the art, the reservoir is often supported on a housing only in the forward direction, for example, on an adapter. The state-of-the-art adapter can be secured on the pump housing in general, supporting the reservoir in the forward direction and connecting the reservoir to a fluid path by means of a connecting needle, for example. An axial external force acting on the adapter may have the effect that the adapter and the reservoir supported on it can move relative to the pump housing due to extremely small bearing plays. However, the plunger of the reservoir is supported on a fixed spindle device, so that the reservoir can move relative to the plunger, and therefore the liquid medication can be dispensed. This disadvantage of the state of the art can be eliminated by the bearing support of the disposable part and/or the reservoir by means of cams on a depository designed on the chassis, wherein this is preferably carried out in the form of a bayonet connection according to the invention.

For the state-of-the-art metering device, specifically that of the first generation, the longitudinal extents and tolerances of the housing must always be taken into account together since the reservoir in this embodiment is supported on the housing by means of the adapter. In these known embodiments, the spindle device is supported on the rear housing bottom and can execute a maximum stroke that is always the same and may correspond to a constant number of motor steps, for example. However, the reservoir is supported on the opposite housing inside wall by means of the adapter, for example. The housing itself has a great fabrication tolerance, special embodiments of the housing which are manufactured by means of plastic injection molding methods, have large tolerances. This is a disadvantage and leads to the result that a residual volume may remain in the reservoir in the case of the maximum housing length. At the minimum housing length, the total stroke of the spindle device is just capable of dispensing the total amount of liquid medication in the reservoir. Using such dimensioning prevents the plunger from running into the wall of the reservoir at the end of the dispensing and thereby triggering a false alarm. This consideration shows that the longitudinal tolerance of the housing has a definitive influence on the remaining volume. The length tolerances of housings can usually amount to several tenths of a millimeter; the remaining volume may turn out to be unfavorably large accordingly. In the state of the art, for example, insulin pumps of the first generation, the residual volume may amount to as much as 5% of the reservoir volume. For a reservoir with a volume of 2 mL and use of U100 insulin, the result may be a residual volume of 10 IU insulin that cannot be utilized, wherein the reservoir holds a maximum of 200 IU insulin. The present invention creates a better solution here. Due to the fact that the bayonet connection is disposed approximately at the same height as the stop for the spindle device, the length tolerance of the housing here no longer has an influence. Only the longitudinal extent of the reservoir and the spindle device have a direct influence on the residual volume of liquid medication after dispensing the medication.

The metering device according to the invention therefore accomplishes the goal of minimizing the residual volume of liquid medication after dispensing the medication. The metering device according to the invention therefore achieves the result of minimizing and reducing the residual volume of liquid medication at the end of dispensing the medication. This is in turn achieved by the fact that the reservoir as a disposable part is supported approximately at the level of the support of the spindle device so that the influence of the length tolerance of the housing can be eliminated. When considered mathematically, this means that the tolerance of the housing does not enter into the calculation in a tolerance analysis. Only the tolerances of the reservoir and the spindle device enter into a so-called tolerance chain. This also means that the component having the greatest tolerance, i.e., the housing length, can be eliminated from the tolerance chain by the design according to the invention, so that a significant advance can be achieved. Combining the at least one disposable part with the reusable part by means of a longitudinal guide and a bayonet connection constitutes a separate invention. It is possible to improve the handling, but the unusable residual volume can also be reduced.

The metering device may have a disposable part, but it may also be formed from a plurality of disposable parts. A design having two disposable parts is very advantageous. For a metering device having two disposable parts the following arrangement is advantageous and recommended. In this particularly interesting embodiment, the first disposable part includes the reservoir and the spindle device disposed on the plunger. The second disposable part may include a base plate and an insertion head. The base plate may have a bottom side, which can be placed flatly on the tissue and is prepared for attachment to the tissue. In this way the metering device according to the invention can be worn directly on the patient's body which his especially advantageous for the user. The insertion head may be fixedly connected to the base plate; an arrangement in which the insertion head is displaceable from a secured starting position into a secured end position against the base plate by applying a minimal deployment force is an advantageous alternative embodiment wherein the direction of travel of the insertion head may be at an angle to the axis of travel of the spindle device. The angle between the axis of travel of the spindle device and the direction of travel of the insertion head is advantageously 90 angular degrees. However, the insertion angle may have less than 90 angular degrees, for example, 20 angular degrees to 60 angular degrees for a flat insertion.

The fluid-carrying connection for subcutaneous administration of the liquid medication is advantageously disposed on the insertion head. In this embodiment variant which is particularly favorable, the insertion head includes a cannula housing with a cannula to be placed in the tissue, a through-channel for the liquid medication leading to the annular and two borders of the through-channel formed by septa, wherein the one septum can be penetrated by a connecting needle disposed on the reservoir and the other septum as well as the cannulas in the starting position can be penetrated by a puncture needle in the direction of travel of the insertion head. Fundamentally, a reversal of the placements of the connecting needle and the septum is also conceivable. For example, the reservoir may have a septum and the insertion head may have the connecting needle for connecting to the reservoir. More advantageous is an arrangement in which the connecting needle is disposed on the reservoir. This arrangement offers the advantage that after connecting the first disposable part to the reusable part, the user can perform a priming in which the spindle device is first moved in reverse to its stop, then can be moved in the direction of advance. As soon as the user observes a droplet of liquid medication emerging from the connecting needle, he can terminate the priming by means of the controller. However, if the insertion head is fixedly connected to the base plate as proposed by said alternative embodiment, then it is advantageous to arrange the connecting needle on the insertion head and to design the reservoir with a septum that can be penetrated by the connecting needle. In this variant the user first connects the first disposable part to the reusable part and then connects the unit formed in this way to the second disposable part so that the entire metering device is prepared for placement on the body. The user can primarily perform a priming, in which the entire fluid-carrying connection is filled with liquid medication. After the priming, the user can apply the entire metering device formed by the two disposable parts and the one reusable part to a location on his body. Then the insertion needle is inserted into the skin first and next the sticky bottom side of the base plate is applied to a flat area of skin and attached there by adhesion.

For linear displacement of the insertion head, the base plate may have guide means. The guide means may be designed advantageously in the form of at least one profiled rail. The at least one rail engages in a guide path. The guide path may advantageously be designed directly on the cannula housing. In addition, to the guidance means, holding means are necessary for holding the insertion head. The holding means are preferably disposed on the base plate. The insertion head can first be held in the secured starting position by the holding means and then held in the secured end position after deployment, wherein the deployment takes place by applying the minimum releasing force, and in the end position, the cannula is placed in the user's tissue. The holding means are preferably formed by at least one flexible snap-hook disposed on the base plate. The at least one snap-hook can engage in two positions on the cannula housing and can secure the cannula housing in the starting position and the end position. The insertion head can also be secured in the starting position by a removable locking element. The locking element facilitates handling for the user and prevents the insertion head from being advanced unintentionally toward the body while attaching the second disposable part to a location on the body. Only when the user has carried out all the handling steps does he remove the locking element. Then the insertion head is operable. The deployment of the insertion head from its upper position into its end position can take place by applying a minimum deployment force. However, the deployment can also be triggered by an auxiliary device, for example, by means of a plunger-spring device. After the deployment, the cannula is in its end position in the subcutaneous tissue of the user. By removing the insertion needle, the cannula for the infusion with mediation fluid is released. The insertion head that has a cannula housing for placement of a cannula and is displaceable toward a base plate may constitute its own invention. The cannula housing may also have a hood as a cover. Both the guidance means and the holding means can act directly on the cannula housing of the insertion head.

The second disposable part preferably has means for accommodating the reusable part connected to the first disposable part. For this purpose, the base plate has at least one linear guide, which serves as a linear guide for at least one longitudinal groove formed on the housing. The linear guide is directed so that in the coupled state the connecting needle of the first disposable part is flush with the first septum. By linear displacement of the housing to an axial stop base formed on the base plate, the septum can be penetrated by the connecting needle. This achieves the result that the connecting needle is guided to penetrate the septum, so that it is possible to avoid such consequences as sealing problems and the like that occur with poor penetration. An embodiment in which the base plate has two separate parallel linear guides that can be inserted at corresponding longitudinal grooves formed on the housing is especially preferred.

In addition, it is advantageous if the first linear guide is disposed laterally on the base plate and if the second linear guide is disposed in parallel to the first linear guide. Therefore, a particularly stable connection between the second disposable part and the reusable part can be formed. In addition, it is advantageous if only one linear guide is first brought to engagement. In order for the user to be able to easily connect the housing to the second disposable part, the housing is first coupled to the first lateral linear guide and then is coupled to the second linear guide by subsequent displacement of the housing along the lateral first linear guide, wherein the housing can rest on the base plate in a flat position. The coupling of the housing to the second disposable part as described here is particularly inventive and may itself constitute a separate invention. The intuitive handling with which the housing is brought to engagement successively on two longitudinal guides disposed in the disposable part and the subsequent displacement, which is guided in a straight line with the associated penetration of the septum by the connecting needle should be emphasized in particular. The proposed connection and coupling between the housing and the second disposable part are intuitive and can be carried out easily by a user. Furthermore, penetration of the septum by the connecting needle, which is guided in a straight line, is possible, so that leakage problems between the septum and the connecting needle can be reduced substantially.

The bayonet connection between the first disposable part and the reusable part is preferably designed as a fixed bearing and the first disposable part is additionally supported on the second disposable part by means of a rotationally secured friction bearing. The friction bearing does not absorb any axial force acting in the forward direction. The rotationally secured friction bearing advantageously has at least one bearing pair formed by a guide and a guide pin that can be inserted into the guide. It is especially advantageous if the guide of the friction bearing is formed on the stop base of the base plate and if the guide pin is disposed on the first disposable part. In bringing the first disposable part into engagement with the second disposable part, only the at least one guide pin is brought into engagement with the at least one guide and subsequently the septum is penetrated by the connecting needle which is guided in a straight line. The first disposable part preferably has two guide pins. In addition, a design in which the housing of the reusable part can be supported on the stop base of the second disposable part is favorable. The proposed bearing in which the first disposable part is supported on the second disposable part by means of a fixed bearing on the reusable part and by means of the friction bearing on the second disposable part is especially advantageous. The fixed bearing secures the first disposable part in the forward direction and in the direction opposite the forward direction so that the first disposable part is not axially displaceable and can also absorb transverse forces. However, the friction bearing between the first disposable part and the second disposable part cannot absorb any axial force, but the friction bearing may be designed so that it can prevent rotation of the first disposable part along its longitudinal axis. In addition, the friction bearing may be designed so that it can absorb forces transversely to the longitudinal axis of the first disposable part. With such an inventive combination for the bearing of the first disposable part, it is possible to achieve the result that the first disposable part is determined statically and is not supported in an overdetermined manner. The friction bearing prevents rotation and absorbs the torque; it also absorbs bearing forces transversely to the longitudinal axis. However, it allows a relative displacement between the first and second disposable parts in which the connecting needle can be displaced axially only relative to the septum. The fluidic connection between the reservoir and the fluid-carrying channel can be preserved. An axial relaxation, i.e., in the forward direction and in the direction opposite the forward direction is achieved by means of the bayonet connection.

In addition, it is advantageous to support the housing of the reusable part on the stop base of the second disposable part. This is especially advantageous because in this way it is possible to achieve the result that external forces acting on the housing, for example, compressive forces in operation lead only to the result that the housing is supported on the stop base. Since the first disposable part is supported with a friction bearing on the second disposable part, the displacement of the second disposable part relative to the reusable part does not result in any displacement of the first disposable part relative to the reusable part. This is especially advantageous because it achieves the result that when the two parts are compressed—the reusable part against the second disposable part—there is no displacement of the plunger relative to a wall of the reservoir, so that liquid medication could be dispensed. In particular in the state of the art, for example, the D-TRONplus pump, the reservoir is supported via an adapter. The adapter can be compressed or displaced toward its axial stop by the user within the context of its bearing play. In doing so, the reservoir is also displaced and moves in reverse relative to the fixed plunger supported by the spindle device, so that the liquid medication can be dispensed. For example, an axial bearing play of 0.01 mm between the adapter and the housing and a plunger cross section of 100 mm² leads to dispensing of 0.1 IU insulin when using U100 insulin. It is clearly apparent here that the bearing by means of a fixed bearing and a friction bearing according to the invention here offers a significant advantage because, with this type of bearing, such an unintentional dispensing of medication can be prevented completely. The bearing of the first disposable part by means of a fixed bearing and a friction bearing thus constitutes another independent invention, which achieves a definite improvement in comparison with the state of the art. In addition, a releasable connection formed by at least one groove and one cam may be present between the reusable part and the second disposable part for securing the insertion head in its end position. By means of this additional connection, it is possible to ensure that the insertion head is held in its end position, in which the cannula is placed in the patient's tissue. In addition, the locking element may be designed so that the reusable part can be attached to the second disposable part only after removing the locking element. Therefore, the connection to be formed from a groove and a cam can be locked by means of the locking element for securing the insertion head in its end position, so that the cam cannot be run into the groove. Only when the locking element has been removed can the cam be run into the groove.

In addition, the metering device should have an axial fixation for an axial fixation of the reusable part on the second disposable part. The axial fixation may be formed from at least one releasable snap-hook connection, for example. The snap-hook connection is preferably formed by a hook formed on the housing and a recess formed on the base plate and provided for the hook. A reversal of the arrangement of the hook and the recess is also conceivable. The hook can engage on an edge of the recess and can secure the reusable part axially but is connected to the first disposable part. In addition, the housing may have an operating head, by actuation of which the flexible base plate can be spread by the housing in a region of the snap-hook connection, to thereby release the snap-hook connection. The flexible base plate can be spread mostly at a right angle to the support on the housing. The operating head may in general be disposed on the housing, even on a disposable part. An arrangement of the operating head on the third housing part is especially advantageous because this is easily replaceable as a gear bottom for repair purposes. The operating head may be designed to be elastic and may have a spreading head designed in the form of a wedge. On actuation, i.e., due to elastic deflection by a force exerted by the user, the wedge-shaped spreading head can act on a dividing line formed by the housing and the base plate to spread the base plate. It is especially advantageous to also form the base plate in the form of a wedge at an engagement site on the spreading head so that mutually facing skewed contact planes of the spreading head and the base plate come to lie parallel to one another, so that the spreading head can separate the base plate from the housing especially well on activation. In addition, it is advantageous if the elastic operating button has a limited deflection. It is possible in this way to achieve the result that the user cannot deflect the operating head beyond a defined reliable extent and cannot damage it in this way. Deflection beyond a defined strain limit can shorten the lifetime of this component. An embodiment in which the metering device has two snap-hook connections to be released by operating buttons is especially advantageous. The operating buttons should be operable simultaneously by hand in this embodiment. To do so, the operating buttons may be disposed on opposing side faces of the housing. The goal here is for the user to be able to operate the metering device with one hand. Bringing the reusable part into engagement with the second disposable part that is glued to the patient's body as well as the release of these parts by activation of the operating heads should take place by hand. The operating buttons are disposed and designed in such a way that the user can release them with his thumb and an index finger. To further secure the metering device, another snap-hook connection may be provided between the housing and the base plate. This connection is preferably released by a minimal axial tensile force on the housing. The user first activates the operating buttons by using one thumb and one index finger and thereby releases the two snap-hook connections. By pulling axially along the longitudinal guide, it subsequently generates the required force to release the snap-hook connection. The snap-hook connection may be formed by just one shallow wedge formed on the housing and a recess that is provided for the wedge on the elastic base plate. The embodiment described here for fixation of the reusable part with the second disposable part by means of two snap-hook connections and one optional snap-hook connection is especially advantageous and constitutes a separate invention. The intuitive handling should be emphasized here, wherein the user can carry out the fixation as well as the release of this fixation by using one hand.

In addition, it is especially advantageous if the support and bearing of the reservoir on the chassis are accomplished not only by the bayonet connection but instead additional means are provided here. At least one support element may be disposed on the chassis for this purpose. In displacement of the reservoir to its depository along the longitudinal guide, the support elements engage in the reservoir. In doing so, the support elements contact the inside wall of the reservoir on their outer circumference and can thus further restrict any radial deflection of the reservoir. The reservoir is subsequently rotated into the bearing slots. In doing so, the cams engage in the bearing slots and thereby form the bayonet connection. The inside wall of the reservoir is then no longer supported on the support elements over the circumference but instead is supported along a contact line. An external force acting on the reservoir is thus partially directed onto the chassis via the bayonet connection, while at the same time a portion of the applied force is absorbed by the support elements along the contact lines. In this way it is possible to achieve a spindle device that is free of transverse forces.

In addition, it should be pointed out that the metering device, in particular the insertion head, may also have a sensor for measuring blood glucose levels. It is advantageous in particular to design the sensor so that the sensor and the fluid-carrying cannula together form one component, so that the user will see only one puncture site.

### BRIEF DESCRIPTION OF THE DRAWINGS

One exemplary embodiment of the invention is described in greater detail below on the basis of the drawings, in which

FIG. 1a shows a metering device according to the invention before being coupled to a completely filled first disposable part, shown in longitudinal section,

FIG. 1b shows the metering device illustrated in FIG. 1a after coupling, shown in a longitudinal section,

FIG. 2a shows a metering device according to the invention before being coupled to a partially filled first disposable part, shown in longitudinal section,

FIG. 2b shows the metering device illustrated in FIG. 2a after coupling, shown in a longitudinal section,

FIG. 2c shows the metering device illustrated in FIG. 2b after a run-up of a spindle device onto its stop, shown in a longitudinal section,

FIG. 2d shows the metering device illustrated in FIG. 2c with a completely extracted spindle device shown in longitudinal section,

FIGS. 3a-3e show the design and assembly of the reusable part consisting of three housing parts shown in 3D,

FIG. 4a shows the first disposable part in a 3D diagram connected to a pull-up rod,

FIG. 4b shows the first disposable part illustrated in FIG. 4a in an exploded diagram,

FIG. 5a shows the second disposable part secured with a locking element in a 3D diagram,

FIG. 5b shows the second disposable part illustrated in FIG. 5a after removing the locking element and before the release of the insertion head, shown in a 3D diagram,

FIG. 5c shows the second disposable part illustrated in FIG. 5b in an exploded diagram,

FIG. 5d shows the second disposable part illustrated in FIG. 5c after release of the insertion head shown in a 3D diagram,

FIG. 5e shows the second disposable part illustrated in FIG. 5d after release of the insertion head shown in a longitudinal section,

FIGS. 6a-6c show the handling steps for connecting the first disposable part to the reusable part shown in a 3D diagram,

FIGS. 6d-6f show the handling steps for the second disposable part in a sectional view transversely to the forward direction,

FIGS. 6g-6j show the handling steps for connecting the reusable part connected to the first disposable part to the second disposable part shown in a 3D diagram,

FIG. 6k shows the metering device illustrated in FIG. 6i in a sectional view transversely to the forward direction, section A-A,

FIG. 6l shows the metering device illustrated in FIG. 6i in a sectional view along the forward direction, section B-B,

FIGS. 7a-7c show the metering device illustrated in FIG. 6i, wherein an operating button is activated here and is not activated in the 3D diagram and is shown in a sectional view,

FIG. 7c shows the metering device illustrated in FIGS. 7a and 7b, wherein the arrangement and the activation of the operating buttons are illustrated here and

FIGS. 8a-8b show an advantageous embodiment of the bearing of the first disposable part on the reusable part.

### DETAILED DESCRIPTION

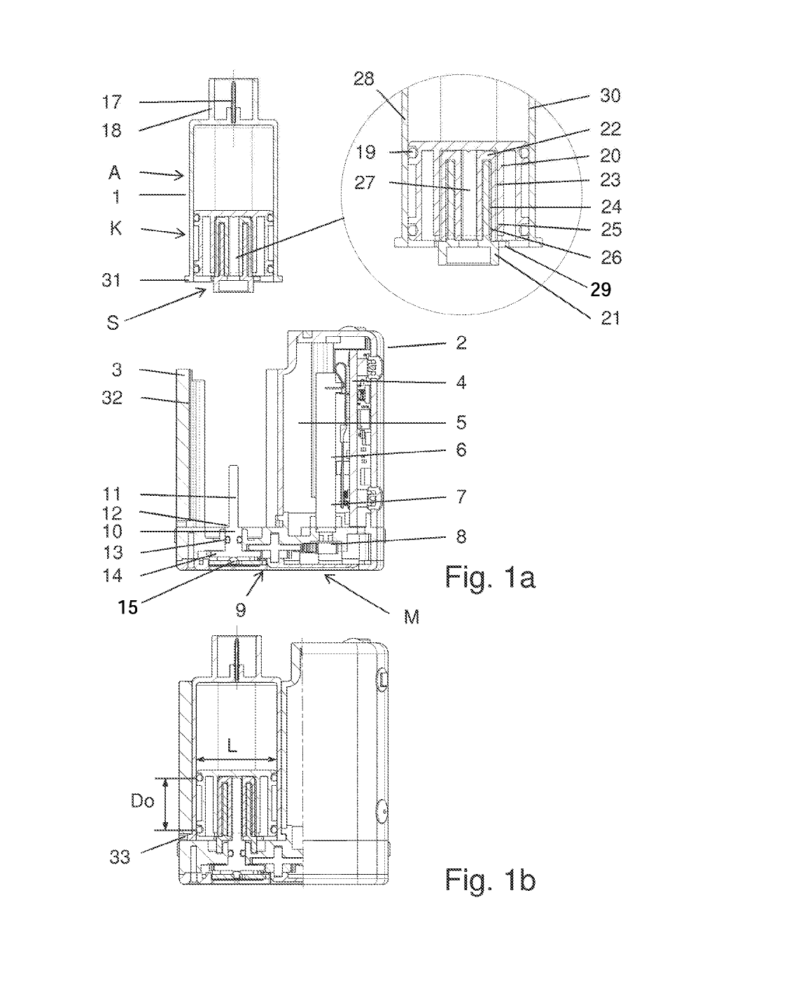

FIG. 1a and FIG. 1b show a metering device D according to the invention. The metering device D according to the invention is formed from disposable parts and one reusable part. The reusable part 2 comprises a housing 3, on which are disposed an electronic circuit board 4 with a controller, a battery 5, a motor 6 and a planetary gear 7 coupled to the motor 6 as well as gearwheels 8 of a spur gear 9 for deflection. A last output wheel 14 of the spur gear 9 is designed with a coupling element via a shaft 10. The coupling element is designed as a driving rod 11, for example, with a rectangular profile, and has a stop 12 for a spindle device S. The output wheel 14 is disposed in the housing 3 so that it is floating and mounted in a straight line wherein a sealing location is formed on the shaft. The sealing location may be formed by a guide for the shaft having small tolerances. In the exemplary embodiment in FIGS. 1a and 1b, the sealing location is in the form of an O-ring 13. The output wheel 14 is support by means of a force sensor 15 on a fixed base.

In FIG. 1a the first disposable part 1 in the form of a reservoir A and the reusable part 2 of the metering device D are shown separately from one another. In a first handling step the user fills the reservoir A. In doing so, the user can fill the first disposable part 1 by means of a pull-up rod 16 as usual. The reservoir A shown in FIG. 1a is suitable for use in an insulin pump. It has a volume of 2 mL and can thus hold 200 IU insulin with a concentration of U100. The reservoir A here has a connecting needle 17, which is provided for connecting a fluid-carrying connection F between the reservoir A and the user. To prevent the user from being injured by the needle, the reservoir A has a protective collar 18 disposed around the needle 17. A plunger K held by means of two O-rings 19 is disposed in the reservoir A.

A spindle device S is disposed on the plunger K itself. This spindle device is designed in the form of a telescoping spindle and consists of two displacement stages 20, 21 and one driving stage 22. The first displacement stage 20 is designed on the plunger K itself and therefore the plunger has an inside thread 23. The second displacement stage 21 has an outside thread 24 and moves only in reverse. The driving stage 22 is formed by two cylindrical sleeves which are connected to one another on one end face. The sleeves are preferably connected to one another on the end face that faces the plunger K. The driving stage 22 has an outside thread 25 on the outer sleeve, forming a first spindle drive with the inside thread 23 of the first displacement stage 20. The outer sleeve additionally has an inside thread 26 which forms a second spindle drive with the outside thread 24 of the second displacement stage 21. The inner sleeve has a profiled elongated hole 27, in which the driving rod 11 can be inserted during the coupling. The spindle drives thus formed are contra-rotating. In the exemplary embodiment shown here, the spindle pitch amounts to 0.5 mm per revolution. In delivery, the driving stage 22 is separated from the second displacement stage 21 wherein a repulsion amounting to 0.5 mm per revolution occurs here. In addition, the first displacement stage 20 is again deflected by the driving stage 22 by another 0.5 mm/revolution. This yields a forward movement for the plunger K as a function of the revolution of the driving stage 22 of 1.0 mm per revolution, for example. The reservoir A is designed to have a noble cross section.

A reservoir wall 28 serves as a twist-proof element for the plunger K and/or for the first displacement stage 20 as well as for the second displacement stage 21. The second displacement stage 21 therefore has radial wings 29 protruding outward and supportable on an inside wall 30. The second displacement stage 21 can also be coupled to the pull-up rod 16. During filling, the user can displace the plunger K beyond the pull-up rod 16. During filling in general, the reservoir A is coupled to a storage tank where the connecting needle 17 is suitable, for example. The plunger K is subsequently displaced and air in the reservoir A is displaced into the storage container. By reverse displacement of the plunger K, liquid medication flows from the storage container into the reservoir A. This process can be repeated several times until the reservoir A has reached the desired filling level and the reservoir A is free of bubbles. In FIG. 1a the reservoir is filled completely and accordingly the plunger K is in its rear position. It should be emphasized here that, although the plunger K is displaced during filling, the spindle device S remains in its starting state during filling. This means that, after filling, the spindle device S is in its defined retracted position, i.e., its starting position from which it can always be moved by a complete total stroke H.

The reservoir A shown in FIG. 1a has two outside cams 31 on the wall 28. For connecting the disposable part 1 to the reusable part 2, the cams 31 are first brought into engagement with two longitudinal guides 32 formed on the housing 3. While in engagement, the disposable part 1 is axially displaceable only along the guides 32. By displacement of the disposable part 1 toward a depository, the spindle device S is coupled to a driving device M. The driving device M comprises the motor 6 together with the planetary gear 7, the spur gear 9 and the output wheel 14 on which the driving rod 11 is disposed as a coupling element for the spindle device S. The driving device M is assigned to the reusable part 2 while the spindle device S is assigned to the disposable part 1.

In concrete terms, in longitudinal displacement of the first disposable part 1, the driving rod 11 is inserted into the elongated hole 27 in the spindle device S. Driving device M and spindle device S are thus coupled for transfer of a drive power to the spindle device S. The rotational drive power is converted by the spindle device S into a translational stroke work for the displacement of liquid medication. By further displacement, the first disposable part 1 reaches its bearing point, at which the cams 31 of the disposable part 1 are rotated into bearing slots 33. In this position, the disposable part 1 is coupled to the reusable part 2. On the one hand the disposable part 1 is fixedly connected to the reusable part 2 and the wall 28 of the reservoir A is fixedly secured in its depository formed on the housing 3. A displacement of the reservoir wall 28 in the forward direction as well as in the direction opposite the forward direction is prevented by the bearing. The bearing by means of two cams 31 engaging in slots 33 corresponds to a bayonet connection.

In addition, after coupling according to FIG. 1b, the driving rod 11 is inserted into the elongated hole 27. Rotating of the driving rod 11 by the driving device M leads to rotation of the driving stage 22 of the spindle device S. After the coupling, a longitudinal play is present in general between the second displacement stage 21 and the stop 12 formed on the driving rod 11. By driving the spindle device S, the second displacement stage 21 moves first in reverse against its stop 12. In approach to the stop 12, a reactive force which is sent by the force sensor 15 to the controller is generated. The controller then stops the motor 6. The reverse movement against the stop 12 corresponds to a stroke H1. The motor 6 that is used may be a brushless dc motor which can be controlled by Hall sensors.

The acknowledgment of a rotor position is made via three Hall sensors offset by 120 angular degrees each. The Hall sensors offset by 120 angular degrees supply six different switching combinations per revolution. The motor 6 may include three partial windings, each of which can be energized in six different conductive phases according to the sensor information, so that the motor has six motor steps per revolution. The current and voltage curves may be in block form. The controller monitors the motor steps via the Hall sensors and can even count the motor steps. The three Hall sensors make it possible to detect a revolution of the motor in six discrete motor steps of 60 angular degrees each. The motor in this exemplary embodiment has an angle resolution of 60 angular degrees. Thus if one knows the number of motor steps from the start to the run-up on the second displacement stage 21 onto its stop 12, then the stroke H1 can be calculated from this. To do so, the number of motor steps n_{Motor} must be multiplied times 60 angular degrees which yields the total motor angle n_{Motor}·60. This total motor angle must subsequently be divided by the step down ratio of the gear i from which the angle of the output wheel

$$\frac{{n}_{Motor} ∀ 60}{i}$$

and/or of the driving stage 22 can be calculated. In addition, the angle of the driving stage 22 can be multiplied by the pitch of the thread p, so that ultimately the stroke H1 in moving in reverse can be calculated. This stroke H1 amounts to

$$\frac{{n}_{Motor} ∀ 60}{i} ∀ p .$$

The stroke H1 can be determined on the basis of the motor steps from the starting position until run-up onto the stop 12. Starting with this known stroke H1, the filling level can also be calculated.

To do so, the stroke H1 is subtracted from the total stroke H of the of the spindle device S thus yielding the stroke H2 which is still available for dispensing the liquid medication. The filling level is therefore obtained from the total reservoir volume V₀ multiplied times the ratio of H2/H. The initial filling level V₁ can thus be calculated with the following formula:

$${V}_{1} = \frac{H - \frac{{n}_{Motor} 60}{i} p}{H} \cdot {V}_{0} = \frac{H - {H}_{1}}{H} \cdot {V}_{0}$$

Starting from the initial filling level V₁ of the reservoir and the motor steps that are also known and can be detected by the controller in dispensing medication, the instantaneous filling level of the reservoir can be calculated. If the filling level drops below a minimum, the user can advantageously be sent an alarm about the pending change in the reservoir. The user can always inquire as to the instantaneous filling level of the reservoir, for example, via a control unit for controlling the metering device, which may be disposed on a blood glucose meter, for example. The smallest metering increment V_{increment} corresponds to the volume ejected in movement of the motor by one motor step. To do so, the stroke is multiplied times the plunger cross-sectional area

$$\frac{π ∀ {D}_{K}^{2}}{4} .$$

The volume of the smallest metering increment therefore corresponds to the following equation:

$${V}_{increment} = \frac{1 \cdot 60}{i} \cdot p \cdot \frac{π \cdot {D}_{K}^{2}}{4}$$

If the volume of the reservoir is divided by the volume of the smallest metering increment, this ultimately yields a constant number of metering increments or motor steps for ejection of a reservoir.

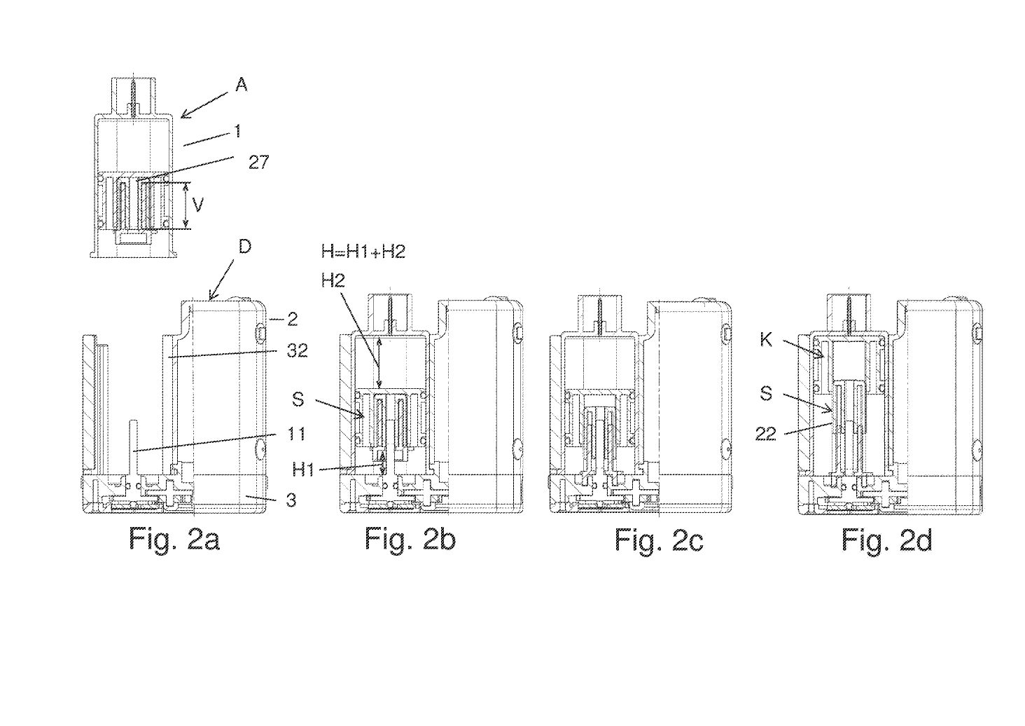

FIG. 2a through FIG. 2d show a sequence, in which a partially filled reservoir A is connected to the reusable part 2 of the metering device D. No fluid-carrying connection F between the reservoir A and an injection site is shown here. Starting from FIG. 2a, the metering device D according to the invention can also accommodate partially filled reservoirs. For the user the handling is like the handling for a completely filled reservoir A. In a first handling step, the partially filled reservoir A is connected to the reusable part 2. The contents of the reservoir A are sent to the depository formed on the housing 3 via the longitudinal guide 32. In doing so, the driving rod 11 is inserted into the elongated hole 27 in the driving stage 22. After coupling according to FIG. 2b, the partially filled reservoir A is coupled to the reusable part 2. To do so, the reservoir A is connected to the housing 3 via the bayonet connection. In partial filling the intersection between the driving rod 11 and the elongated hole 27 is less than in complete filling. The exemplary embodiment shown in FIG. 2b has a length approximately equal to the distance of travel V of a spindle drive for the driving rod 27 starting from the stop 12. According to this the minimum initial filling amounts to 50% of the complete filling. This means that a reservoir A, which is only 50% full can still be coupled. In doing so, there is also a coupling between the driving rod 11 and the driving stage 22.

It can also be seen in FIGS. 2a and 2b that after filling, the spindle device S is in its retracted position, as presupposed by the invention. After coupling, the spindle device S only moves in reverse against its stop 12 and in doing so travels the stroke H1. The control unit can calculate the stroke H1 from the motor steps and from this it automatically determines the initial filling level. Starting from this initial filling level, it is not always possible to calculate the instantaneous filling level for the dispensing because the control unit always knows the additional motor steps taken in dispensing the liquid medication. At the end of this dispensing, the plunger K is in its extracted position as shown in FIG. 2d. The telescopic spindle device S is now completely extracted. Between the driving rod 11 and the driving stage 22, only a minimum intervention is available.

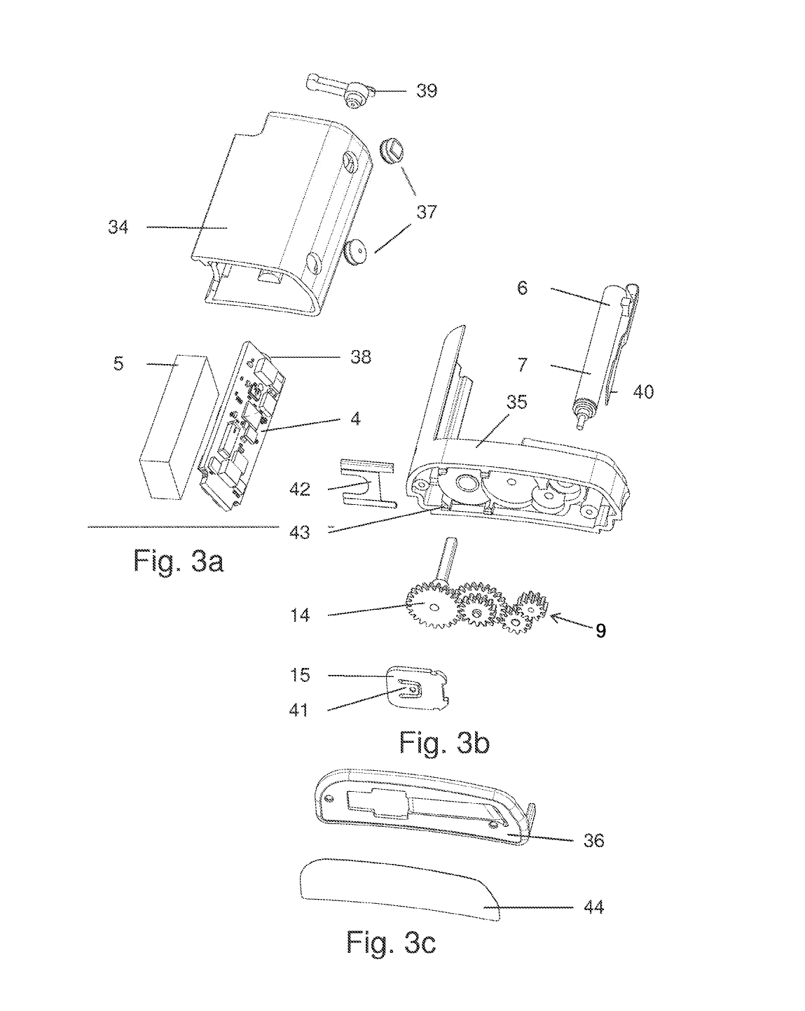

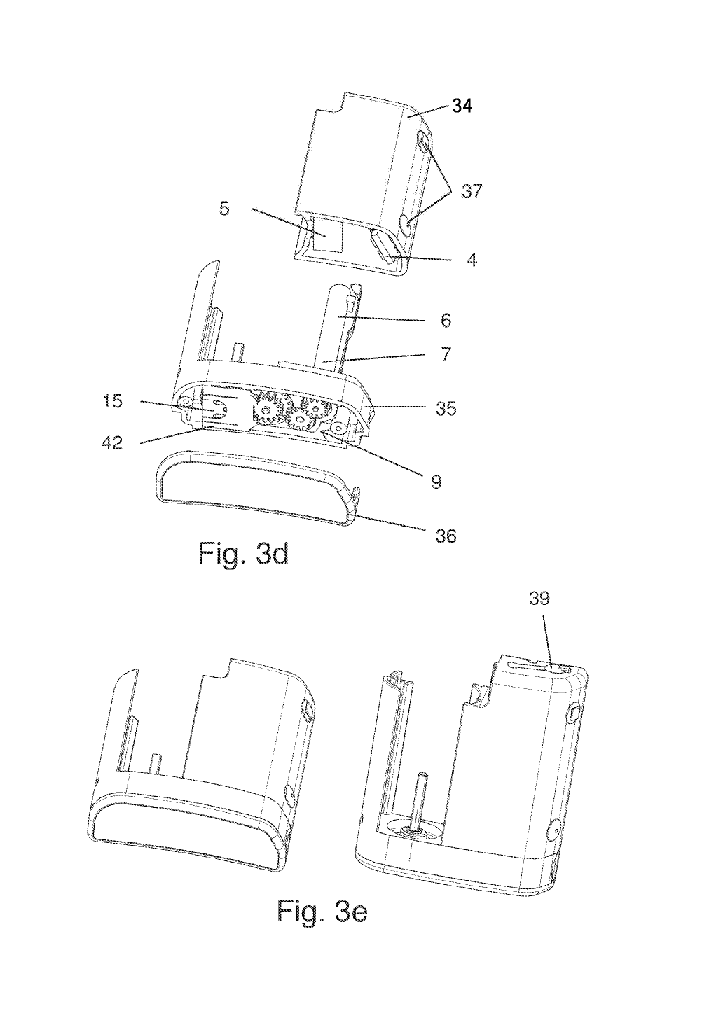

In FIG. 3a through FIG. 3e, the reusable part 2 of the metering device D is illustrated. The reusable part 2 preferably consists of three housing components. The battery 5 may be disposed on a first housing component 34. For example, this battery may be a reachable battery which can be charged via a traditional charger for a cell phone, for example. In addition, the electronic circuit board 4 is disposed on a first housing component 34. For example, two pushbuttons 37 for manual operation are disposed directly on this circuit board. Likewise, a charging plug 38 for the battery is disposed directly on the electronic circuit board 4. In order for the first housing part 34 to be leak-proof, access to the charging plug formed on the housing 3 can be closed and sealed by means of an elastic strap 39. The electronic circuit board 4 has at least one microprocessor for controlling the metering device D. The motor 6 is connected to the electronic circuit board 4 via a FlexPrint 40, i.e., a flexible electrical connection. The electronic circuit board 4 may also have means for issuing alarms as well as, for example, Bluetooth communication means. In general the data exchange of the metering device D as well as the programming and control are possible via external communication devices. For example, the metering device D can be controlled via a smartphone or via a blood pressure meter. However, the user can also perform the control of the metering device D manually. The two operating buttons 37 disposed on the pump are available for this purpose. The metering device D can exchange data with a computer, a smartphone or a blood pressure meter via a Bluetooth communication interface, for example. Communication with a blood pressure sensor worn on the body, in particular an implanted sensor, is also possible.

FIG. 3b shows the second housing component 35. This component is designed as a chassis 35 which holds the essential components of the driving device M. The motor 6 with the repositioned planetary gear is disposed on the chassis. By means of the additional spur gear 9, the last output wheel 14 of the gear is driven. The last output wheel 14 of the gear is driven via the additional spur gear 9. The last output wheel is connected to the driving rod as a coupling element. The spur gear is installed from a lower side of the chassis. The last output wheel 14 is supported here on the force sensor 15. The force sensor 15 itself has a bending bar 41 wherein the force deflection of the output wheel 14 acts on the bending bar 41 and generates an electric signal that is proportional to the deflection and sends it to the control unit via a connecting line (not shown). The force sensor 15 and the output wheel 14 are secured on the chassis 35 by means of a plate 42. The plate 42 may be inserted laterally at two linear guides 43 and can hold the force sensor 15 and the output wheel 14 in the installed position.

FIG. 3c illustrates the third housing component 36 which is simply a gear bottom. The gear bottom 36 may be attached adhesively or screw connected to the chassis 35. It is advantageous here that no force can occur that acts on an interface between the gear bottom 36 and the chassis 35, so that this connection can be sealed well and can have a long lifetime. In addition, the gear bottom 36 can be closed with a cover plate 44 that can be glued on.

According to FIG. 3d, the first housing part 34 and the chassis 35 are preinstalled first. This means that the battery 5 and the electronic circuit board 4 are installed first on the first housing part 34 and the two operating buttons 37 and the strap 39 for the battery plug are also installed. The chassis 35 as well as the components disposed on the chassis 35 are also preinstalled. In doing so the motor 6, the planetary gear 7, the spur gear 9 together with the output gearwheel 4 are installed. Then the output wheel 14 is secured axially via the force sensor 15 and the plate 42. Ultimately the first housing component 34 is installed on the chassis 35 which can be accomplished by means of a screw connection or an adhesive connection. In addition, the gear bottom 36 is fastened onto the chassis 35.

FIG. 3e illustrates the completely installed reusable part 2. The simple installation and force-free accommodation of the gear bottom 36 and the first housing part 34 on the chassis 35 are all advantageous.

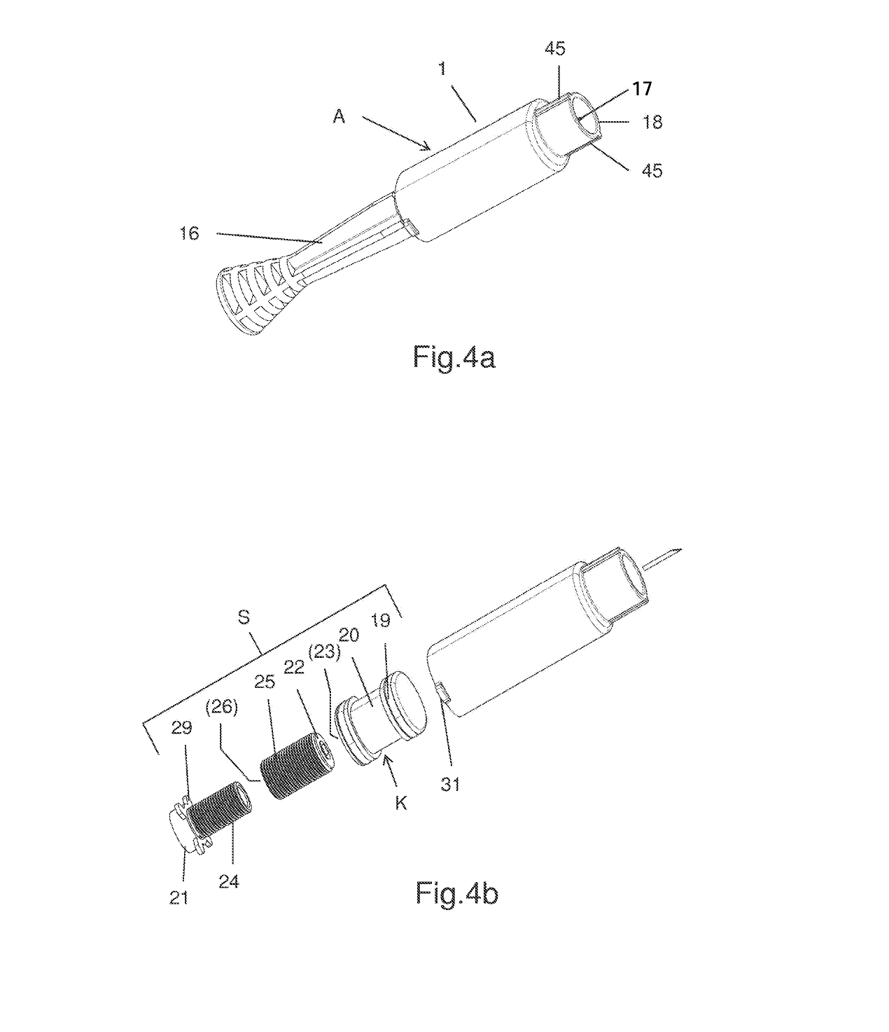

FIGS. 4a and 4b show the first disposable part 1. The first disposable part 1 is the reservoir A. The reservoir A has the reservoir wall for receiving the liquid medication. The exemplary embodiment here has the connecting needle 17 by means of which the reservoir A can be connected to a downstream fluid-carrying connection F. In order for the user not to be injured by the connecting needle 17, the needle 17 is surrounded by the safety collar 18. The safety collar 18 has two guide pins 45 by means of which the reservoir A can be mounted in a rotationally secured manner.

In addition, the reservoir A has the plunger K. The plunger K itself is part of the spindle device S and therefore has the inside thread 23 with which the outside thread 25 of the driving stage 22 is engaged. The driving stage 22 consists of two sleeves which are connected to one another on the side facing the plunger K. The outside thread 24 of the second displacement stage is engaged with the inside thread 26 of the driving stage 22. In assembly, for example, the driving stage 22 is screwed into the plunger K and then the second displacement stage 21 is screwed into the driving stage 22. The two spindle drives are contra-rotating, which means that the one spindle drive has a right-hand thread, while the other spindle drive has a left-hand thread. The plunger K has two sealing sites where O-rings 19 may be disposed. The plunger K together with the spindle drive S disposed on the plunger K is displaced into the reservoir A after assembly. The second displacement stage 21 is secured to prevent rotation by means of the wings 29, which protrude radially outward and can be supported on the inside wall 30. The first displacement stage 20 and the second displacement stage 21 are secured against rotation so that the rotational securing is accomplished via the reservoir wall 28. The second displacement stage 21 can also be connected to the pull-up rod 16. It is advantageous here that the telescopic spindle device S can be formed from only two additional components which can be connected to one another and installed by simple assembly. These additional components include the driving stage 22 and the second displacement stage 21. Moreover the plunger K can be connected to the pull-up rod 16 via the second displacement stage 21.

The filling takes place by displacement of the plunger K via the pull-up rod 16 which is an accustomed handling action for the user. In addition, the plunger K has a good stability in the reservoir A. One measure of the quality of the bearing of the plunger K in a reservoir A for preventing tumbling is the length ratio L/Do, where L denotes a longitudinal axis of the oval plunger cross section and Do denotes the distance between sealing sites on the plunger K. In addition, the reservoir A has two cams 31 by means of which the reservoir A can be mounted on the reusable part 2. The cams 31 form part of a bayonet connection 31, 33. The telescopic spindle device S consists of the two displacement stages 20, 21 and the driving stage 22, which is driven by the driving rod 11. In the case of the telescopic spindle device S, the first displacement stage 20 moves in the forward direction with the stroke H2, while the second displacement stage 21 moves only in reverse with the stroke H1. The driving stage 20, which is driven to rotate here, can be moved axially in the forward direction as well as in reverse, i.e., in the direction opposite the forward direction.

The second- and third-generation devices described in the state of the art have only one displacement stage and one driving stage and belong to the category of one-stage spindle devices having a spindle drive. With these devices, for example, a spindle rod may be driven by a driving device and may serve as a driving stage. At the same time, however, the spindle rod also serves as a displacement stage because it abuts against a fixed spindle nut during its drive. In this embodiment, the spindle rod is both a driving stage and a displacement stage. In another state-of-the-art embodiment variant, the spindle rod is fixedly connected to the plunger and therefore serves only as a displacement stage. The spindle nut is in an axially fixed position but can also be driven to rotate by a driving device. In this variant, the spindle nut serves as a driving stage of the spindle device but it does not perform any displacement work on its own.

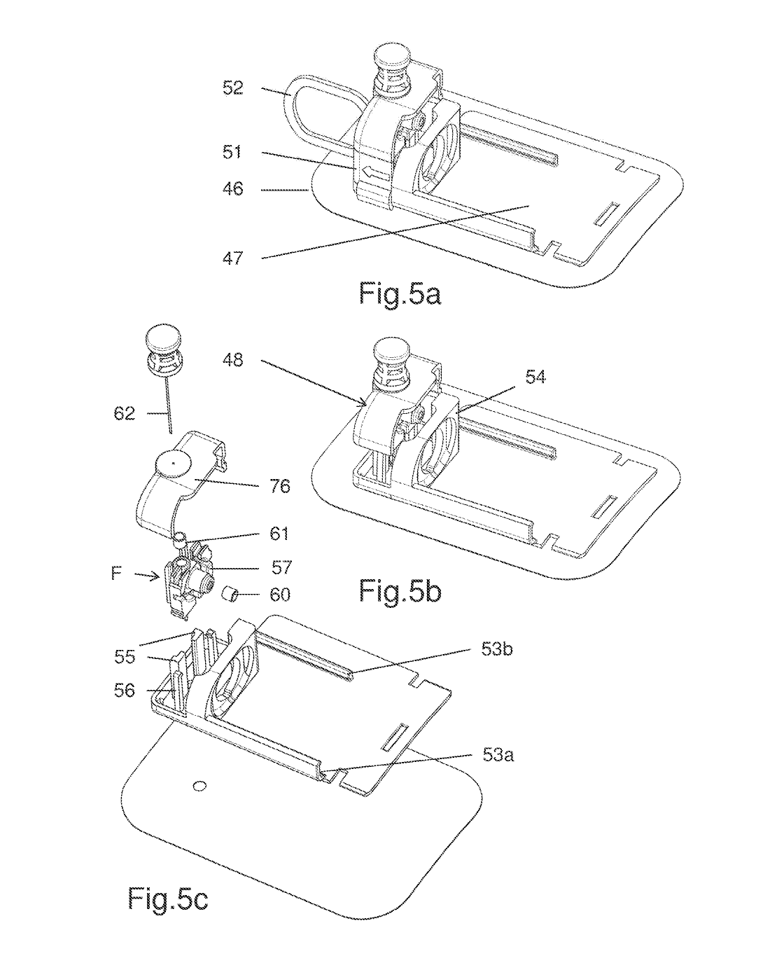

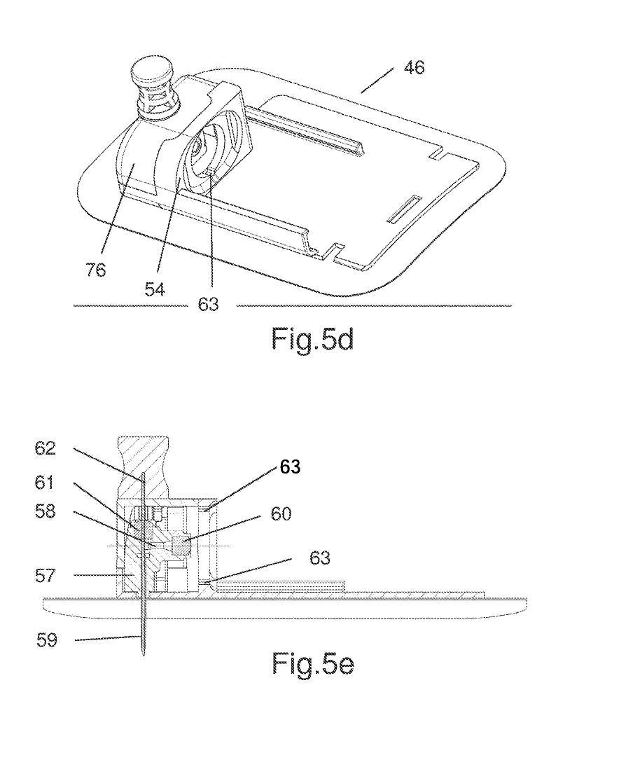

The second disposable part 46 is described with reference to FIGS. 5a to 5e. The second disposable part 46 has a base plate 47 and an insertion head 48. The base plate 47 has a bottom side 49 which can be place flatly on the tissue, which is prepared for fixation on the tissue to which it can be applied over a flat area. The bottom side 49 may be designed with a self-stick function. The user first removes a protective film 50 and then sticks the second disposable part 46 on a disinfected body location via the self-stick bottom side 49. Before the user can use the second disposable part 46, which has been stuck on his skin, he must remove a locking element 51. The locking element 51 has a handle 52 for this purpose by means of which the user can grip the locking element 51 well. By pulling, the user removes the locking element 51 according to FIGS. 5a and 5b.

The second disposable part 46 is shown in an exploded diagram in FIG. 5c. In addition to the self-stick bottom side 49, the second disposable part 46 has a base plate 47. Two longitudinal guides 53 for receiving the reusable part 2 are provided on the base plate 47. In addition, the base plate 47 has a stop base 54 for the reusable part 2. In the forward direction the reusable part 2 is supported on the stop base 54. In addition, the base plate comprises guide means in the form of profiled rails 55 for the angular displacement of the insertion head 48. The profiled rails 55 have an L-shaped cross section here. To allow the insertion head 48 to be held securely in two positions, the base plate has restraint means. The restraint means are designed here as two flexible snap-hooks 56 which are disposed on the base plate and can be engaged in two positions on the insertion head. In addition, the insertion head 48 has a cannula housing 57. The cannula housing 57 is the core part of the insertion head 48, on which the fluid-carrying connection F between the reservoir A and the user is formed. The fluid-carrying connection F consists of a channel 58 which is formed in the cannula housing 57 and leads to a soft cannula 59. The soft cannula 59 may be injection molded on the cannula housing 57 or glued in place there. The fluid-carrying channel 58 bordered by two septa 60, 61. The one septum 60 can be penetrated by the connecting needle 17 disposed on the reservoir A. In the starting position, the other septum 61 and the cannula 59 can be penetrated by a puncture needle 62 in the direction of travel of the insertion head 48.

Starting from the starting position in FIG. 5b the user actuates the insertion button 48. When a minimum releasing force is applied, the two snap-hooks 56 are released from their locks and they release the insertion head 48. The insertion head then travels at a high speed along its guide 55 toward its end position. In the end position, the insertion head 48 is again secured by the snap-hooks 56.

Furthermore, the cannula 59 is in the user's tissue as shown in FIGS. 5d and 5e. FIG. 5e shows the fluid-carrying through-channel 58 in a longitudinal section delimited by the septa 60, 61. In the direction of travel of the insertion head 48, both the septum 61 and the cannula 59 disposed on the cannula housing 57 are punctured by the puncture needle 62. Furthermore, guides 63 which are provided for the guide pins 45 of the reservoir A can be seen on the stop base 54. The guides 63 and the guide pins 45 together from a rotationally secured friction bearing by means of which the reservoir A is supported by the friction bearings on the stop base 54. Furthermore, such a bearing allows the septum 60 to be penetrated by the connecting needle 17 in a straight line when connecting the reusable part 2 to the second disposable part 46. By removing the puncture needle 62, the cannula 59 is released for the subcutaneous infusion with liquid medication. In general the insertion head 48 may have a hood 76 which can serve as part of the housing 3. The hood 76 may be fastened to the cannula housing 57.

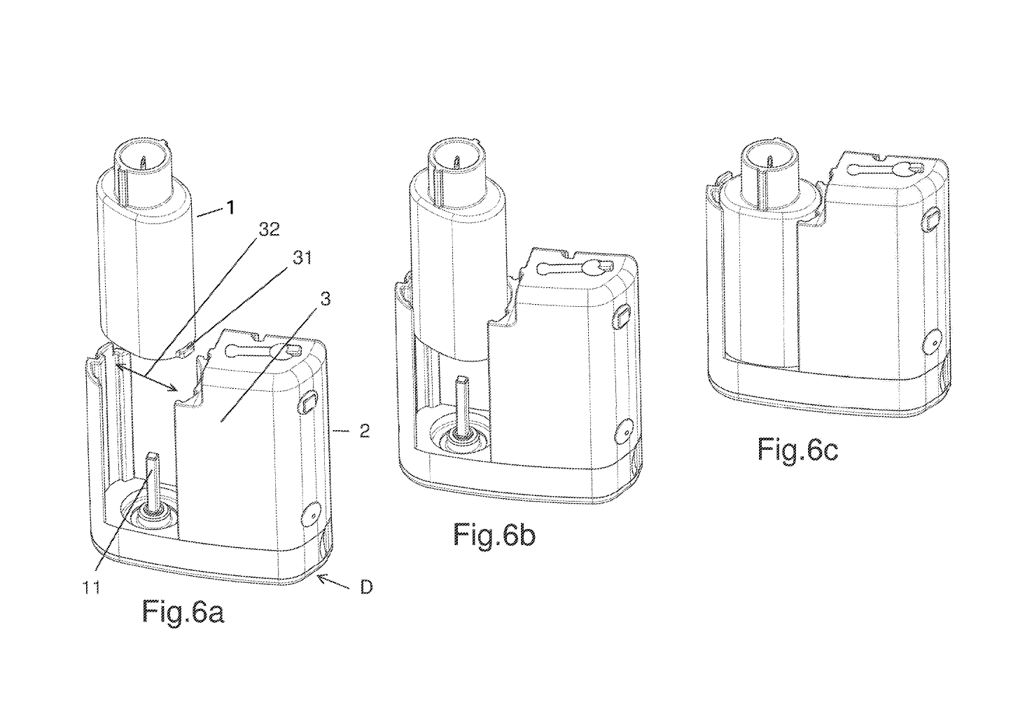

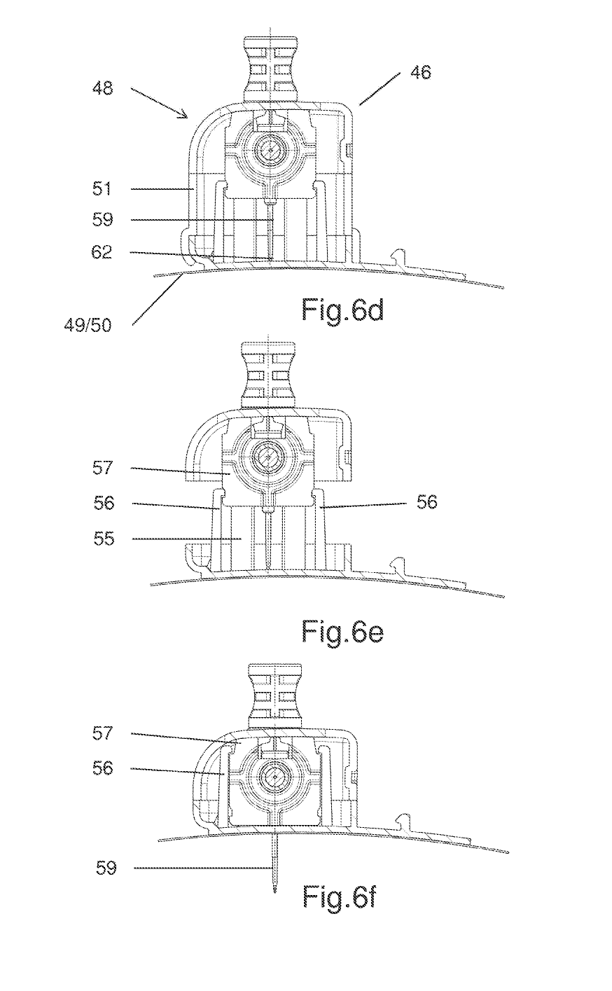

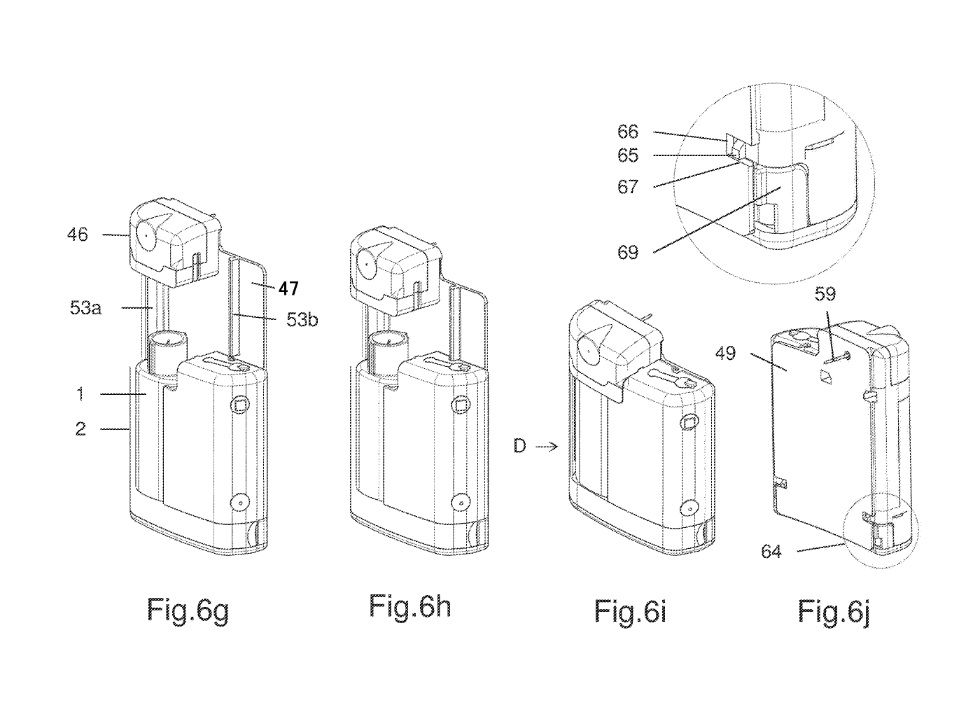

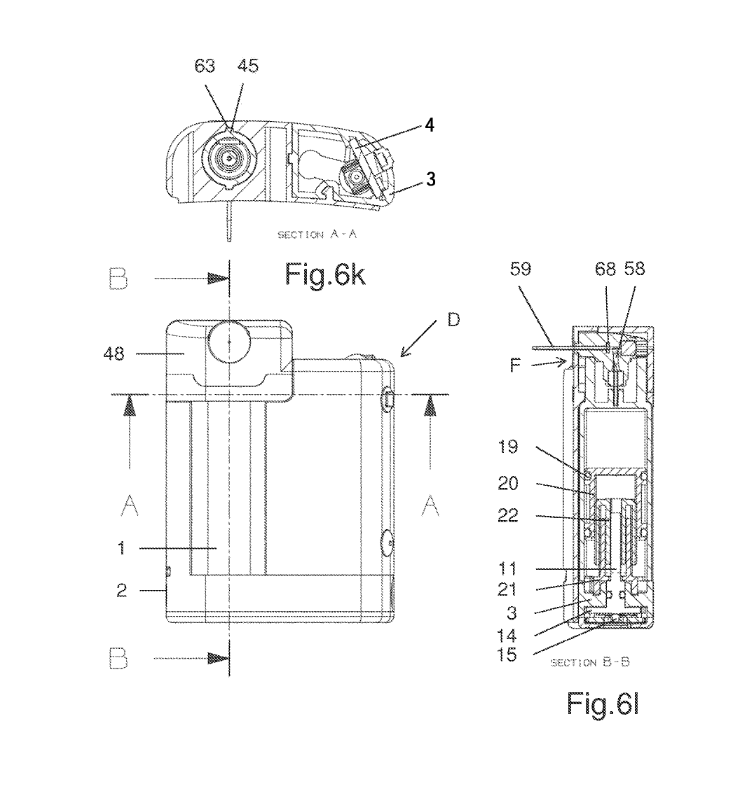

The handling of the metering device D for the user is discussed with reference to FIGS. 6a to 6j. In a first handling step, the first disposable part 1 is accommodated by means of the longitudinal guide 32 formed on the housing 3 and is displaced in a straight line by means of this guide. In displacement the driving rod 11 is inserted into the axial longitudinal hole 27 in the spindle device S. By further displacement, the first disposable part 1 is sent via the longitudinal guide 32 formed on the housing 3 to its depository on the housing 3. In the process, the cams 31 disposed on the first disposable part 1 are rotated into the bearing slots 33 formed on the housing. In this way the user couples the first disposable part 1 to the reusable part 2. Now the first disposable part 1 is secured in both the forward direction and in the direction opposite the forward direction. In addition, the driving device M is coupled to the spindle device S via the driving rod 11. The user can then carry out a priming. The spindle device S first moves in reverse toward its stop 12 since the spindle device S is usually in its retracted position after filling, regardless of whether the reservoir A is partially or completely filled. The run-up onto the stop 12 is detected by the controller, which then stops the motor 6.

The handling of the second disposable part 46 is illustrated in FIGS. 6d to 6f. The user first removes the protective film from the bottom side 49 and sticks the self-stick bottom side 49 of the second disposable part 46 on a suitable location on his body. In order to prevent the user from being injured, the cannula 59 is secured in the insertion head 48 and is not disposed so that it is accessible. The design of the insertion head 48 is very advantageous in particular for users who suffer from a needle phobia because the user cannot visually perceive the puncture needle 62 or the cannula 59 in the basic position because they are disposed in the interior of the insertion head 48. Subsequently, the locking element 51 is removed, so that the insertion head 48 is ready for insertion of the cannula 59.

In FIG. 6e, it is easy to see how the two lateral snap-hooks 56 hold and secure the cannula housing 57 in the starting position. By applying a minimal releasing force, the snap-hooks 56 are deflected until they are no longer in engagement with the cannula housing 57. Without being secured, the cannula housing 57 then moves along the guide means 55 at a high speed toward its end position due to the force acting on it. In the end position, the snap-hooks 56 are again in engagement with the cannula housing 57 and secure the cannula housing 57 in the new position. In addition, the cannula 59 is now placed in the tissue. In FIGS. 6g to 6j, the reusable part 2, which is connected to the first disposable part 1 is then connected to the second disposable part 46. The reusable part 2 which is connected to the first disposable part 1 can be brought into engagement with at least one longitudinal guide 53 formed on the base plate 47 as a linear guide and then subsequently displaced axially along the at least one longitudinal guide 53a. In the case of axial displacement along the longitudinal guide 53a, the reusable part 2, which is connected to the first disposable part 1, is brought into engagement with a second longitudinal guide 53b. By axial displacement to the stop base 54 which is formed on the base plate 47, the first disposable part 1 is guided with the second disposable part 46 and is connected in a rotationally secured manner. The guide pins 45 of the first disposable part 1 then engage the guides 63 on the second disposable part 46. By additional displacement, the septum 60 is finally penetrated linearly by the guided connecting needle 17.

FIGS. 6i and 6j show the metering device D completely. FIG. 6j shows the complete metering device D from the bottom side 49. The cannula 59 protrudes here from the base plate 47 and may extend into the tissue of the user's body, starting from the base plate 47. In insulin pump therapy, the depth of penetration of the cannula 59 is between 5 and 12 mm. In the exemplary embodiment, the puncture angle amounts to 90 angular degrees. The puncture angle may also amount to between 10 and 60 angular degrees, and the cannula housing 57 can travel on an inclined plane.

In addition, FIG. 6j shows a snap-hook connection 64 between the reusable part 2 and the base plate 47 in detail. This connection is formed by a hook 65 which is designed on the housing 3 and a recess 66 on the base plate 47. In the coupled state according to FIG. 6j, the hook 65 engages in the recess 66 and secures the reusable part 2 on the base plate 47. The base plate 47 is designed to be elastic in the area of the recess 66. In addition, it is pointed out that the insertion head 48 can also be released by means of an additional devise. In the case of such a device, for example, a ram may be prestressed by a spring. When the ram is released, it travels and strikes the insertion head 48. In doing do, the insertion head 48 is moved by means of the ram from its starting position into its end position, and the cannula 59 is thereby placed in the tissue.

FIG. 6k shows a section of the metering device D across the forward axis. The section here is made at the height of the insertion head 48 on the stop base 54. This diagram shows clearly how the reservoir A is supported on the stop base 54. For this purpose, the reservoir A has two guide pins 45, which engage in the guides 63 and the stop base 54. Such a bearing is especially advantageous because it achieves the result that transverse forces can be absorbed by the bearing. Furthermore, this prevents the reservoir A from rotating along the forward axis. However, the depository does not absorb any forces acting in the forward direction. A displacement between the housing 3 and the second disposable part 46, i.e., the stop base 54 does not result in a displacement of the reservoir A. This therefore prevents such displacement from causing the medication to be dispensed unintentionally. In the state of the art, a similar displacement of the adapter relative to the housing causes unintentional dispensing of medication because the reservoir A is supported on the adapter with these devices. Due to the fact that the reservoir A is not supported on the stop base 54 but instead is only frictionally supported on the stop base 54, said unintentional dispensing of medication according to the state of the art can be prevented. Furthermore, FIG. 6k shows the support of the electron circuit board 4 in the housing 3 on a guide.

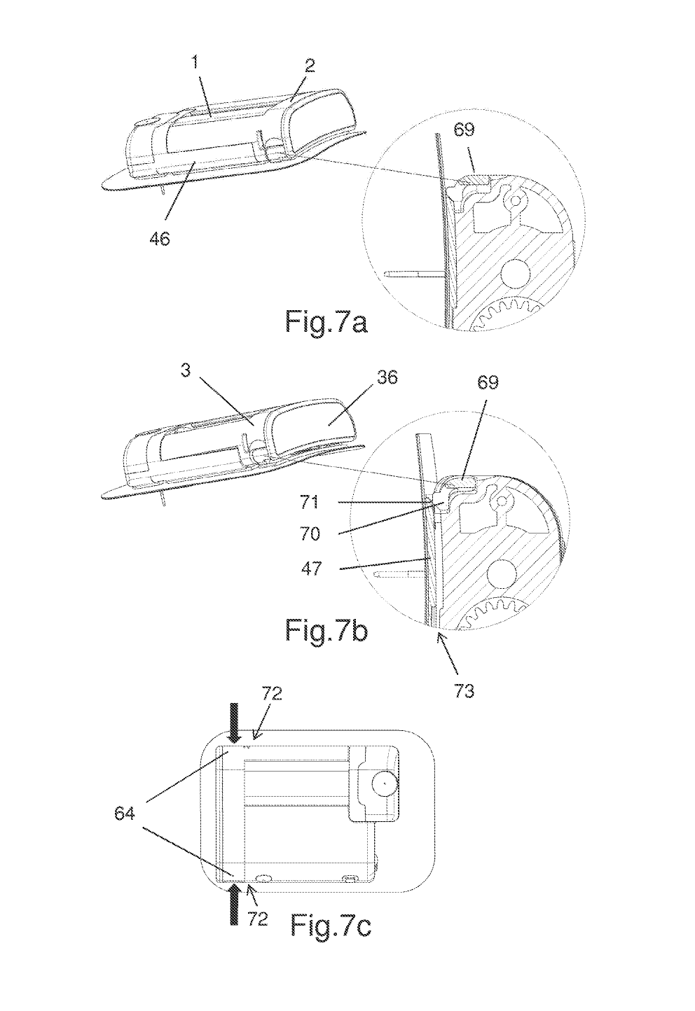

The section of FIG. 6l is along the forward axis and shows the design of the metering device D in a longitudinal section. The output wheel 14 of the driving device M is supported via the force sensor 15. The output wheel 14 is connected via the shaft to the driving rod 11 which drives the driving stage 22 of the spindle device S rotationally. FIG. 6l shows a partially filled reservoir A. Priming has already taken place here so the second displacement stage 21 is supported on its stop 12 which is formed on the driving rod 11. The plunger K itself is designed as the first displacement stage 20 and is held in the reservoir A by means of two O-rings 19. The connecting needle 17 of the first disposable part 1, i.e., the reservoir A protrudes through the septum 60 of the cannula housing into the fluid-carrying channel 58. The channel 58 has a deflection of 90 angular degrees and opens into the cannula 59. The cannula 59 itself has a collar 68, which is especially suitable for sheathing at the time of its manufacture. The puncture needle 62 here has already been removed by the user so that the fluid-carrying connection F is filled by priming with liquid medication starting from the reservoir A up to the cannula outlet. The metering device D is ready for dispensing the medication to the user. The snap-hook connection 64 in FIG. 6j is shown again in detail in FIGS. 7a to 7c. The reusable part 2 is preferably secured axially on the second disposable part 46 by means of at least two releasable snap-hook connections 64 formed on the housing 3 and the base plate 47. When the snap-hook connection 64 is released a flexible operating button 69 is operated and deflected as illustrated in FIG. 7b. The operating button 69 has a spreading head 70. If the operating button 69 is deflected by the user, then the spreading head 70 causes the base plate 47 to spread away from the housing 3, thereby releasing the snap-hook connection 64 between the base plate 47 and the housing 3. Both the spreading head 70 and the base plate 47 may be designed in a wedge shape, so that the base plate 47 can be spread well at the engagement location. The spreading head 70 and the base plate 47 may each have inclined planes 71 here, so that good engagement on the base plate 47 by the spreading head 70 is possible. The operating button 67 is preferably part of the housing 3, in particular part of the third housing component 36.

According to FIG. 7c, it is provided that the metering device D has two snap-hook connections 64, which are to be operated by means of lateral operating buttons 69 disposed on opposing side faces 72 of the housing 3 and are operated with one hand at the same time, in particular with a thumb and an index finger. Due to this arrangement according to FIG. 7c, it is possible to ensure that accidental release of a snap-hook connection 64 does not result in release of the housing 3 from the base plate 47. In order to separate the housing 3 from the base plate 47, the two snap-hook connections 64 must always be operated and released simultaneously by the user. In addition, a third snap-hook connection 73 can be provided. This may in turn be disposed between the housing 3 and the base plate 47. This third snap-hook connection 73 can be released only by deflection of the base plate 47 and therefore requires a minimal tensile force on the housing 3. In its release, the user therefore proceeds as follows. Using the index finger and thumb, for example, the user releases the two side hook connections 64. Finally, the third snap-hook connection 73 is released by pulling axially, so that the housing 3 can be separated from the base plate 47 along the longitudinal guide 53.

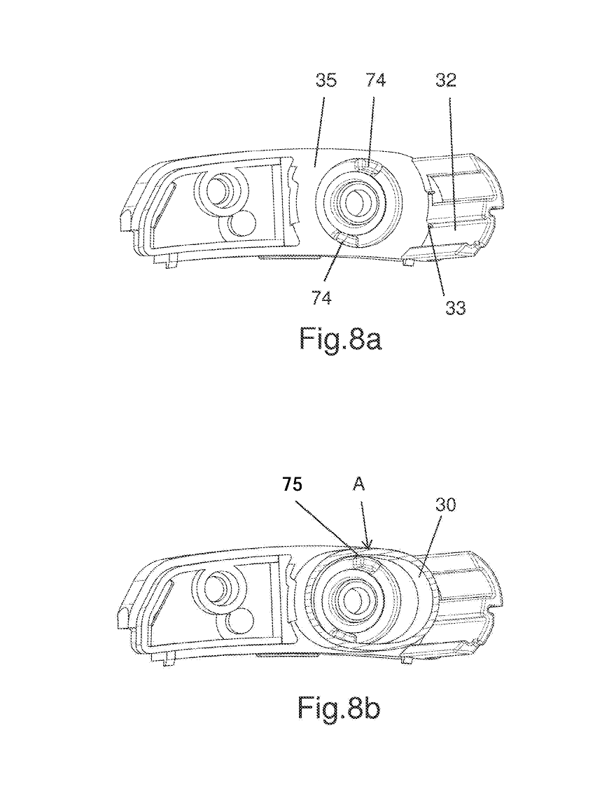

FIGS. 8a and 8b show a particularly advantageous means of support for the reservoir A on the chassis 35. To do so, at least one support element 74 disposed on the chassis 35. In displacement of the reservoir A to its depository along the longitudinal guide 32, the support elements 74 engage in the reservoir A. In doing so, the support elements 74 come in contact with the inside wall 30 of the reservoir A along their outer circumference and thereby prevent any radial deflection of the reservoir A. The reservoir A is subsequently rotated into the bearing slots 33. The cams 31 then engage in the bearing slots 33 and thus form the bayonet connection 31, 33, as already discussed above. The inside wall 30 of reservoir A is then no longer supported on the support elements 74 over the circumference but instead is supported only along a contact line 75. An external force acting on the reservoir A is thus absorbed in part by a bearing slot 33 on the chassis 35 of the bayonet connection. At the same time, some of the external force is directed onto the support elements 74 over the contact lines 75.

### LIST OF REFERENCE NOTATION

S Spindle device
M Driving device
A Reservoir
K Plunger
F Fluid-carrying connection
D Metering device
H Total stroke of spindle device
H1 Reverse stroke
H2 Forward stroke
V Travel distance of a spindle drive
L Longitudinal axis of the plunger cross section
Do Length between sealing sites on the plunger
1 First disposable part
2 Reusable part
3 Housing
4 Electronic circuit board
5 Battery
6 Motor
7 Planetary gear
8 Gearwheels
9 Spur gear
10 Shaft
11 Driving rod
12 Stop
13 O-ring
14 Output wheel
15 Force sensor
16 Pull-up rod
17 Connecting needle
18 Protective collar
19 O-ring
20 First displacement stage
21 Second displacement stage
22 Driving stage
23 Inside thread of first displacement stage
24 Outside thread of second displacement stage
25 Outside thread of driving stage
26 Inside thread of driving stage
27 Elongated hole
28 Reservoir wall
29 Wing
30 Inside wall
31 Cam
32 Longitudinal guide
33 Bearing slot
34 First housing component
35 Second housing component, chassis
36 Third housing component, gear bottom
37 Pushbutton, operating button
38 Charging plug
39 Strap
40 FlexPrint
41 Bending bar
42 Plate
43 Linear guide
44 Cover sheet
45 Guide pin
46 Second disposable part
47 Base plate
48 Insertion head
49 Bottom side
50 Protective film
51 Locking element
52 Handle
53a Longitudinal guide
53b Longitudinal guide
54 Stop base
55 Rail
56 Snap-hook
57 Cannula housing
58 Channel
59 Soft cannula
60 Septum
61 Septum
62 Puncture needle
63 Guide
64 Snap-hook connection
65 Hook
66 Recess
67 Edge
68 Collar
69 Operating button
70 Spreading head
71 Inclined plane
72 Side faces on the housing
73 Third snap-hook connection
74 Support element
75 Contact line
76 Hood

1. A metering device for administering liquid medication to the body of a user wherein the liquid medication is dispensable in a metered manner for the purpose of an infusion or an injection from a reservoir (A) by the advance of a plunger (K) held in the reservoir (A), the metering device (D) additionally comprising:
a) a reusable part (2) comprising a housing (3), a control unit and a driving device (M) wherein the driving device (M) has a coupling element for transfer of a drive power to a spindle device (S) for advancing the plunger (K), and the coupling element is fixedly connected to an output wheel (14) of the driving device (M) and has an axial stop (12) for the spindle device (S);
b) at least one disposable part (1) comprising the reservoir (A) and the plunger (K) which is held in the reservoir and can be advanced by the spindle device (S), wherein the spindle device (S) is disposed on at least one disposable part (1) and has at least one spindle drive with a path of movement (V) per spindle drive wherein the spindle device (S) of the disposable part (1) can be coupled to the reusable part (2) via the coupling element;
c) a fluid-carrying connection (F) between the reservoir (A) and the user and
d) a force sensor (15) disposed on the reusable part (2) which measures a reactive force of the spindle device (S) which serves as a measure of the pressure in the reservoir (A),
wherein
i) the metering device is designed so that the spindle device (S) of the disposable part (1) is in the retracted state after a partial or complete filling of the reservoir (A) and thus a defined total stroke (H) of the spindle device (S) 2 [sic] which is always constant can be traveled,
ii) the coupling element is supported axially with a friction bearing and the output wheel (14) is supported on a fixed base via the force sensor (15) wherein because of the fixed connection between the coupling element and the output wheel (14) when the spindle drive (S) comes to rest against the stop (12) the reactive force of the spindle device (S) acting axially on the stop (12) is directed to the force sensor (15) via the output wheel (14) so that there is a direct transfer of force from the stop (12) via the output wheel (14) to the force sensor (15).

2. The metering device according to claim 1, characterized in that a sealing site is disposed between the output wheel (14) and the coupling element.

3. The metering device according to claim 1 or 2, characterized in that the metering device is designed so that after coupling a recently-filled disposable part (1) and the reusable part (2), the spindle device (S) can be extended against the stop (12) to eliminate a longitudinal play in the direction opposite the forward direction and/or can be extended when the spindle device (S) comes to rest against the stop (12) in the forward direction.

4. The metering device according to any one of claims 1 to 3, characterized in that the coupling element is designed as a profiled driving rod (11) with the stop (12) for the spindle device (S).

5. The metering device according to claim 4, characterized in that the driving rod (11) of the reusable part (2) can be inserted into an axial elongated hole (27) in the spindle device (S) of the disposable part (1) in coupling between the at least one disposable part (1) that is partially or completely filled and the reusable part (2), and the disposable part (1) can be uncoupled from the reusable part (2) by pulling out the driving rod (11) from the axial elongated hole (27).

6. The metering device according to any one of claim 4 or 5, characterized in that the length of the driving rod (11) starting from the stop (12) is equal to or greater than the distance of travel (V) of a spindle drive.

7. The metering device according to any one of claims 1 to 6, characterized in that the driving device (M) has the same direction of rotation for extending it in reverse and for extending it in forward direction.

8. The metering device according to any one of claims 1 to 7, characterized in that the spindle device (S) is disposed on the plunger (K) and is displaceable together with the spindle device (S) in filling the plunger (K).

9. The metering device according to any one of claims 1 to 8, characterized in that the spindle device (S) can always be extracted by the same total stroke (H) for each new reservoir (A), regardless of whether the reservoir (A) is filled partially or completely.

10. The metering device according to claim 9, characterized in that the total stroke (H) is comprised of a reverse stroke (H1) for eliminating the longitudinal play and a stroke (H2) directed in the forward direction for administering the liquid medication.

11. The metering device according to any one of claims 1 to 10, characterized in that the driving device (M) comprises a motor (6) and a gear (7, 9) driven by the motor (6), wherein a last gearwheel is designed as the output wheel (14).

12. The metering device according to any one of claims 1 to 11, characterized in that the housing (3) of the reusable part (2) is formed from three housing parts (34, 35, 36), wherein a first housing part (34) accommodates at least the control unit and/or a power source (5), a second housing part (35) is designed as a chassis (35) and accommodates at least the motor (6), the gear (7, 9) and the coupling element which is designed as a driving rod (11), and a third housing part (36) is designed as a gear bottom (36).

13. The metering device according to claim 12, characterized in that the base for the force sensor (15) is formed by a plate (42) that can be fastened onto the chassis (35).

14. The metering device according to any one of claim 12 or 13, characterized in that the chassis (35) accommodates the first (34) and third housing parts (36).

15. The metering device according to any one of claims 1 to 14, characterized in that the at least one disposable part (1) and the reusable part (2) can be connected to one another via a bayonet connection.

16. The metering device according to claim 15, characterized in that the bayonet connection is formed on the chassis (35).

17. The metering device according to any one of claim 15 or 16, characterized in that the bayonet connection is disposed at approximately the height of the stop (12) for the spindle device (S) with respect to an axial axis of movement for the spindle device (S).

18. The metering device according to any one of claims 15 to 17, characterized in that the housing (3) has an axial longitudinal guide (32) for the at least one disposable part (1) by means of which the spindle device (S) is guided and can be coupled with the coupling element of the reusable part (2) and by means of which the disposable part (1) is guided and can be supplied to a depository for the bayonet connection.

19. The metering device according to claim 18, characterized in that the axial longitudinal guide (32) is formed by two grooves directed toward one another and the at least one disposable part (1) has two cams (31), each engaging in a groove.

20. The metering device according to any one of claims 1 to 19, characterized in that the metering device (D) has two disposable parts (1, 46).

21. The metering device according to claim 20, characterized in that the first disposable part (1) comprises the reservoir (A) and the spindle device (S) disposed on the plunger (K), and the second disposable part (46) comprises a base plate (47) and an insertion head (48).

22. The metering device according to claim 21, characterized in that the base plate (47) has a bottom side that is prepared for fixation on the tissue and can be placed on an area of tissue.

23. The metering device according to any one of claim 21 or 22, characterized in that the insertion head (48) is fixedly connected to the base plate (47).

24. The metering device according to any one of claim 21 or 22, characterized in that the insertion head (48) is displaceable in a straight line from a secured starting position into a secured end position against the base plate (47) by applying a minimal releasing force, and the direction of travel of the insertion head (48) is at an angle to the axis of movement of the spindle device (S).

25. The metering device according to any one of claims 21 to 24, characterized in that the fluid-carrying line (F) is disposed on the insertion head (48) for subcutaneous administration of the liquid medication, and the insertion head (48) comprises a cannula housing (57) with a cannula (59) that can be placed in the tissue, a through-channel (58) for the liquid medication leading to the cannula and two borders of the through-channel (58) formed by septa (60, 61), wherein the one septum (60) can be penetrated by a connecting needle (17) disposed on the reservoir (A), and the other (61) as well as the cannula (59) are penetrated by a puncture needle (62) in the direction of travel of the insertion head (48) in the starting position.

26. The metering device according to any one of claim 24 or 25, characterized in that the base plate (47) has guide means for the linear displacement of the insertion head.

27. The metering device according to claim 26, characterized in that the guide means are designed in the form of at least one profiled rail (55).

28. The metering device according to any one of claims 24 to 27, characterized in that the base plate (47) has restraint means by means of which the insertion head (48) can be held in the secured starting position first and then after being released can be held in the secured end position wherein the release is accomplished by applying the minimal releasing force and in the end position the cannula (59) is placed in the user's tissue.

29. The metering device according to claim 28, characterized in that the restraint means comprise at least one flexible snap-hook (56) that is disposed on the base plate (47), and the at least one snap-hook (56) can be engaged in two positions on the cannula housing (57) and the cannula housing (57) can thus be secured in the starting position and in the end position.

30. The metering device according to any one of claims 24 to 29, characterized in that the insertion head (48) can be secured in the starting position by a removable locking element (51).

31. The metering device according to any one of claims 25 to 30, characterized in that the cannula (59) is released for infusion with the liquid medication by removing the puncture needle (62).

32. The metering device according to any one of claims 21 to 31, characterized in that the second disposable part (46) has means for accommodating the reusable part (2) that is connected to the first disposable part (1).

33. The metering device according to claim 32, characterized in that the base plate (47) has at least one linear guide (53) which serves as a linear guide (53) for the at least one longitudinal groove formed on the housing (3), and in the coupled state, the connecting needle (17) of the first disposable part (1) is flush with the first septum (60), and the septum (60) can be penetrated by the connecting needle (17) by linear displacement of the housing (3) on an axial stop base (54), which is formed on base plate (47).

34. The metering device according to claim 33, characterized in that the base plate (47) has two linear guides (53a, 53b).

35. The metering device according to claim 34, characterized in that the first linear guide (53a) is disposed on the side of the base plate (47), and the second linear guide (53b) is disposed parallel to the first linear guide (53a).

36. The metering device according to claim 35, characterized in that the housing (3) can be coupled to the first linear guide (53a) on the side first and by subsequent displacement of the housing (3) along the first lateral linear guide (53a), wherein the housing (3) sits flatly on the base plate (47) to which the second linear guide (53b) can be coupled.

37. The metering device according to any one of claims 20 to 36, characterized in that the bayonet connection (31, 33) is formed between the first disposable part (1) and the reusable part (2) as a fixed bearing, and the first disposable part (1) can be supported on a second disposable part (46) by means of a rotationally secured friction bearing.

38. The metering device according to claim 37, characterized in that the friction bearing does not absorb any axial force acting in the forward direction.

39. The metering device according to any one of claim 37 or 38, characterized in that the rotationally secured friction bearing has at least one bearing pair formed by a guide (63) and a guide pin (45) that can be inserted into the guide (63).

40. The metering device according to claim 39, characterized in that the guide (63) of the friction bearing is formed on the stop base (54) of the base plate (47), and the guide pin (45) is disposed on the first disposable part (1).

41. The metering device according to claim 39 or 40, characterized in that when bringing the first disposable part (1) into engagement with the second disposable part (46), the at least one guide pin (45) can first be brought into engagement with the at least one guide (63) and subsequently the septum (60) can be penetrated by the linearly guided connecting needle (17).

42. The metering device according to any one of claims 21 to 41, characterized in that, between the reusable part (2) and the second disposable part (46), a releasable connection formed from at least one groove and one cam is provided for securing the insertion head in its end position.

43. The metering device according to any one of claims 33 to 42, characterized in that the housing (3) of the reusable part (2) is supported on stop base (54) of the second disposable part (46).

44. The metering device according to any one of claims 21 to 43, characterized in that at least one releasable snap-hook connection (64) is provided for an axial fixation of the reusable part (2) on the second disposable part (46).

45. The metering device according to claim 44, characterized in that the snap-hook connection (64) is formed by a hook (65) provided on the housing (3) and a recess (66) formed for the hook (65) on the base plate (47), wherein the hook (65) can be hooked on an edge (67) of the recess (66).

46. The metering device according to any one of claim 44 or 45, characterized in that the housing (3) has an operating button (69), the operation of which causes the flexible base plate (47) to be spread in a region of the snap-hook connection (64) by the housing (3), so that it is largely perpendicular to the support on the housing (3) and the snap-hook connection (64) can be released in this way.

47. The metering device according to claim 46, characterized in that the operating button (69) is provided on the third housing part (36).

48. The metering device according to any one of claim 46 or 47, characterized in that the operating button (69) is designed to be elastic and has a spreading head (70) designed in the form of a wedge, and on actuation of the wedge-shaped spreading head (70), the operating button acts on the dividing line formed by the housing (3) and the base plate (47) to spread the base plate (47).

49. The metering device according to claim 48, characterized in that the base plate (47) is also designed in a wedge shape at a site of action of the spreading head (70) so that skewed contact planes (71) of the spreading head (70) and the base plate (47) that are facing one another are also in parallel with one another.

50. The metering device according to any one of claims 46 to 49, characterized in that the elastic operating button (69) has a limited deflection.

51. The metering device according to any one of claims 46 to 50, characterized in that the snap-hook connection (64) to be released by operating buttons (69) are provided for the metering device (D), and the operating buttons (69) can be operated by hand simultaneously, wherein the operating buttons (69) are disposed on opposing lateral surfaces (72) of the housing (3).

52. The metering device according to any one of claims 44 to 51, characterized in that an additional snap-hook connection (73) is provided between the housing (3) and the base plate (47), wherein this can be released by a minimum axial tensile force on the housing (3).
